(19) United States
(12) Patent Application Publication
(10) Pub. No.: US 2018/0200000 A1
(43) Pub. Date: Jul. 19, 2018

(54) SHAPE CALCULATING APPARATUS

(71) Applicant: OLYMPUS CORPORATION, Tokyo (JP)

(72) Inventors: Koichi TAKAYAMA, Koganei-shi (JP); Hiromasa FUJITA, Hachioji-shi (JP); Ken SATO, Hachioji-shi (JP)

(73) Assignee: OLYMPUS CORPORATION, Tokyo (JP)

(21) Appl. No.: 15/869,161

(22) Filed: Jan. 12, 2018

Related U.S. Application Data
(63) Continuation of application No. PCT/JP2015/070295, filed on Jul. 15, 2015.

(51) Int. Cl. A61B 34/20; A61B 1/07; A61B 1/00; A61B 1/005; G01B 11/16; G01D 5/353

(52) U.S. Cl. CPC A61B 34/20; A61B 1/07; A61B 1/00006; A61B 2034/2061; G01B 11/16; G01D 5/35351; A61B 1/005

(57) ABSTRACT

A shape calculating apparatus includes a light source, an optical fiber provided with detection targets. The detection targets have mutually different light absorption spectra to decrease a quantity of light propagated by the fiber in accordance with a bend shape of the fiber. The apparatus also includes a light detector to detect light quantity information at wavelengths included in the light absorption spectra, a calculator to execute a calculation relating to a shape of each detection target based on the light quantity information. The apparatus further includes a setting change unit to change, with respect to each of the wavelengths, a dynamic range of at least either an intensity of light input to the optical fiber or an electric signal generated by the detector.

### CROSS REFERENCE TO RELATED APPLICATIONS

This application is a Continuation Application of PCT Application No. PCT/JP2015/070295, filed Jul. 15, 2015, the entire contents of which are incorporated herein by reference.

### BACKGROUND OF THE INVENTION

### 1. Field of the Invention

The present invention relates to a shape calculating apparatus that calculates a shape of each of detection targets based on light quantity information detected with respect to a wavelength corresponding to each detection target.

### 2. Description of the Related Art

Japanese Patent No. 4714570 discloses an endoscope shape detection probe that bends together with a scope as one piece and detects a shape of the scope. This detection probe includes a light modulator to change a light quantity in accordance with a curvature, as a detection target provided in a fiber for curvature detection. The detection probe with this structure is capable of detecting the shape of the scope based on the intensity or wavelength of light that has been modulated by the light modulator and based on the distance between the light modulator and an output end of the fiber for curvature detection.

Japanese Patent No. 4714570 also discloses that providing detection targets corresponding mutually different wavelength components are provided in the fiber for curvature detection allows detecting not only a shape of a part of the scope, but also shapes of various parts along a desired length.

### BRIEF SUMMARY OF THE INVENTION

A shape calculating apparatus according to an aspect of the present invention includes a light source configured to emit light, an optical fiber disposed in a structure body that is a shape calculation target, and configured to guide light emitted from the light source, detection targets provided in the optical fiber along a longitudinal direction of the optical fiber. The detection targets have mutually different light absorption spectra. The detection targets are configured to absorb the light propagated by the optical fiber in accordance with a bend shape of the optical fiber and to decrease a quantity of light. The shape calculating apparatus further includes a light detector configured to detect light quantity information at wavelengths included in the light absorption spectra of the detection targets, the light quantity information relating to the light that is propagated by the optical fiber and the light quantity of which is decreased by the detection targets, a calculator configured to execute, based on the light quantity information, a calculation relating to a shape of each of the detection targets, and a setting change unit configured to change, with respect to each of the wavelengths included in the light absorption spectra, a dynamic range of at least either an intensity of light that is input to the optical fiber or an electric signal generated by the light detector based on light that is output from the optical fiber.

Additional objects and advantages of the invention will be set forth in the description that follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing a schematic configuration of a shape calculating apparatus according to a first embodiment of the invention.

FIG. 2 is a cross-sectional view of a light guide at a part where a detection target is provided.

FIG. 3A is a view showing a light transmission quantity at a time when the light guide is not bent.

FIG. 3B is a view showing a light transmission quantity at a time when the light guide is bent toward a side opposite to the side where the detection target is provided.

FIG. 3C is a view showing a light transmission quantity at a time when the light guide is bent toward the side where the detection target is provided.

FIG. 4 is a graph showing a light absorption spectrum of each detection target.

FIG. 5 is a block diagram showing a functional configuration of a processor of the shape calculating apparatus of the first embodiment and peripheral parts of the processor.

FIG. 6 is a time chart of sequentially changing the setting of the light intensity of a light source, in order to execute a sequential change of the dynamic range of light intensity of light that is input to a sensor unit as an example of a variable quantity setting change.

FIG. 7 is a time chart of sequentially changing the setting of the exposure time of a light detector, in order to execute a sequential change of the dynamic range of an electric signal that is generated by the light detector, as an example of the variable quantity setting change.

FIG. 8 is a time chart of sequentially changing the setting of the gain of sensitivity of the light detector, in order to execute a sequential change of the dynamic range of the electric signal that is generated by the light detector, as an example of the variable quantity setting change.

FIG. 9A is a view showing a detection signal of each wavelength acquired by the light detector in accordance with a fixed synchronization signal before a change, in changing a synchronization signal of the light detector as an example of the variable quantity setting change.

FIG. 9B is a view showing a detection signal of each wavelength acquired by the light detector in accordance with a synchronization signal after the change in accordance with a required wavelength, in changing the synchronization signal of the light detector as the example of the variable quantity setting change.

FIG. 10 is an operational flowchart of the shape calculating apparatus according to the first embodiment.

FIG. 11 is a view showing a relationship between the shape of the light guide and a detection signal by the sequential variable quantity setting change.

FIG. 12 is a block diagram showing a functional configuration of a processor of a shape calculating apparatus of a second embodiment of the invention and peripheral parts of the processor.

FIG. 13 is an operational flowchart of the shape calculating apparatus according to the second embodiment.

FIG. 14A is a view showing a detection signal before a variable quantity setting change at a time when the detection signal of the light detector exceeds an upper-limit threshold.

FIG. 14B is a view showing a detection signal after the variable quantity setting change at a time when the detection signal of the light detector exceeds the upper-limit threshold.

FIG. 15A is a view showing a detection signal before a variable quantity setting change at a time when the detection signal of the light detector decreases below a lower-limit threshold.

FIG. 15B is a view showing a detection signal after the variable quantity setting change at a time when the detection signal of the light detector decreases below the lower-limit threshold.

FIG. 16 is a block diagram showing a functional configuration of a processor of a shape calculating apparatus of a third embodiment of the invention and peripheral parts of the processor.

FIG. 17 is an operational flowchart of the shape calculating apparatus according to the third embodiment.

FIG. 18A is a view showing a detection signal before a range change by a reference voltage change of an AD converter as an example of the variable quantity setting change.

FIG. 18B is a view showing a detection signal after the range change by the reference voltage change of the AD converter as the example of the variable quantity setting change.

FIG. 19 is a block diagram showing a functional configuration of a processor of a shape calculating apparatus of a fourth embodiment of the invention and peripheral parts of the processor.

FIG. 20 is an operational flowchart of the shape calculating apparatus according to the fourth embodiment.

FIG. 21A is a view showing a detection signal before a setting change in an example of optimal detection signal acquisition by variable quantity setting changes.

FIG. 21B is a view showing a detection signal after changing the setting of exposure time of the light detector from the setting of FIG. 21A, in the example of optimal detection signal acquisition by variable quantity setting changes.

FIG. 21C is a view showing a detection signal after further changing the setting of exposure time of the light detector from the setting of FIG. 21B, in the example of optimal detection signal acquisition by variable quantity setting changes.

FIG. 21D is a view showing a detection signal after changing the setting of light intensity of the light source from the setting of FIG. 21C, in the example of optimal detection signal acquisition by variable quantity setting changes.

FIG. 22 is a block diagram that schematically shows the configuration of an endoscope apparatus in which the shape calculating apparatus according to any one of the embodiments is mounted.

### DETAILED DESCRIPTION OF THE INVENTION

Embodiments for implementing the present invention will be described hereinafter with reference to the accompanying drawings.

### First Embodiment

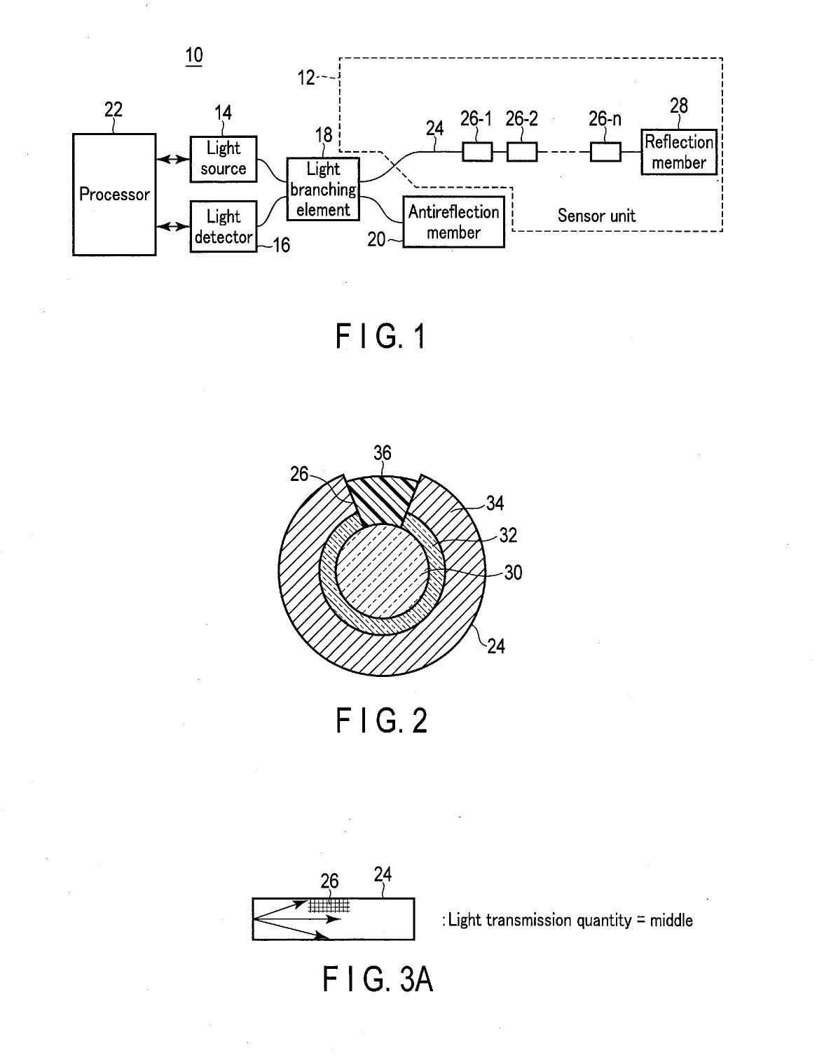

As illustrated in FIG. 1, a shape calculating apparatus 10 according to the first embodiment is composed by a sensor unit 12, a light source 14, a light detector 16, a light branching element 18, an antireflection member 20, and a processor 22. The sensor unit 12 is composed by a light guide 24, an n-number of detection targets 26 (first detection target 26-1, second detection target 26-2, . . . , n-th detection target 26-n), and a reflection member 28.

The light source 14 emits light of necessary wavelength characteristics (e.g. white light) for the shape calculating apparatus 10. As the light of necessary wavelength characteristics for the shape calculating apparatus 10, use can be made of light of a laser diode (LD), an LED, a lamp, etc., or light that a fluorescent material is caused to emit by such light. The light of necessary wavelength characteristics for the shape calculating apparatus 10 is produced by a combination of such light. The light branching element 18 is composed by, for example, a fiber coupler, a half-mirror, or a beam splitter. The light branching element 18 causes light that is emitted from the light source 14 to enter one end of the light guide 24. When the light branching element 18 is a fiber coupler, the light source 14 includes a lens system or the like that converges light to cause it to enter a fiber of the fiber coupler. When the light branching element 18 is a half-mirror or a beam splitter, the light source 14 includes a lens system or the like that collimates light into a parallel beam of light. Besides, when return light affects an output as in the case of a laser diode, the light source 14 includes an isolator or the like.

The light guide 24 guides light that has entered the one end of the light guide 24 by the light branching element 18 to the other end, and then radiates it from the other end. The reflection member 28 reflects light radiated from the other end of the light guide 24, and then causes it to enter the other end of the light guide 24 once again. Thereby, the light guide 24 guides light that has entered the other end to the one end, and then radiates it from the one end. The light branching element 18 inputs light radiated from the one end of the light guide 24 to the light detector 16. The light detector 16 detects quantities of light of predetermined wavelengths in the input light, and then outputs light quantity information, which is a relationship between the wavelengths and light quantities, to the processor 22.

The antireflection member 20 is used in order to prevent light that has not entered the light guide 24 from returning to the light detector 16.

Here, the light guide 24 is disposed to extend along the longitudinal axial direction of a structure body, for instance, an insertion section of an endoscope, the curvature information of which is to be detected by the shape calculating apparatus 10. The light guide 24 has such flexibility as to bend in accordance with the bend shape of the structure body.

Concretely, the light guide 24 can be composed by an optical fiber. FIG. 2 shows a cross-sectional structure in a radial direction, which is a direction perpendicular to the longitudinal axial direction of the optical fiber. To be more specific, the optical fiber is composed by a core 30, which exists at the center and guide light, a clad 32, which is provided around the core 30 and stably confines light in the core 30, and a jacket 34, which protects the core 30 and clad 32 from a physical impact and thermal impact.

Incidentally, the light guide 24 is not limited to the optical fiber, and may be composed by a light guide.

Besides, the light guide 24 is provided with detection targets 26 (first detection target 26-1, second detection target 26-2, . . . , n-th detection target 26-n) with mutually different light absorption spectra at locations corresponding to positions of the structure body where the curvature information is to be detected. Here, the curvature information is information of a direction of bending and a magnitude of bending.

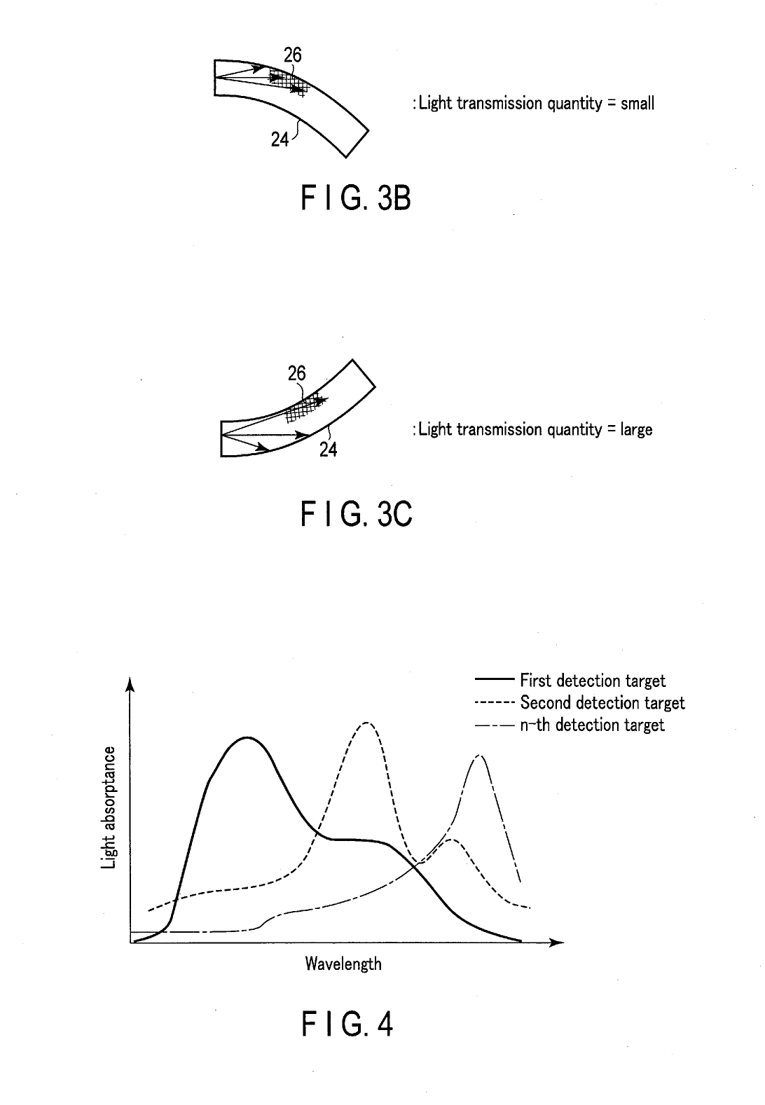

If the curvature of the light guide 24 is varied, the magnitude of the quantity of light guided in the light guide 24 varies. FIG. 3A, FIG. 3B, and FIG. 3C are schematic views showing light transmission quantities corresponding to the bending of the light guide 24. FIG. 3A shows a light transmission quantity at a time when the light guide 24 is not bent. FIG. 3B shows a light transmission quantity at a time when the light guide 24 is bent toward a side opposite to the side where the detection target 26 is provided. FIG. 3C shows a light transmission quantity at a time when the light guide 24 is bent toward the side where the detection target 26 is provided. As shown in FIG. 3A, FIG. 3B, and FIG. 3C, the light transmission quantity varies in the following order. The light transmission quantity is largest when the light guide 24 is bent toward the side where the detection target 26 is provided. The light transmission quantity is next largest when the light guide 24 is not bent. The light transmission quantity is smallest when the light guide 24 is bent toward the side opposite to the side where the detection target 26 is provided. Thus, the amount of bending in the detection target 26 can be detected by measuring the light intensity of a light signal that is output from the light guide 24. In addition, since the position in the radial direction of the light guide 24 where the detection target 26 is provided, that is, the direction of the detection target 26, is already known, the direction of bending can also be understood. Based on the direction of bending and the amount of bending, the curvature information can be detected.

For example, as shown in FIG. 2, the detection target 26 is formed as follows. The jacket 34 and clad 32 are removed at a desired position in the longitudinal axial direction of the light guide 24, so that a part of the core 30 is exposed. A detection target material 36 is formed on the exposed part of the core 30 so as to have such a thickness that the light guide 24 can restore to the original shape. The detection target material 36 is composed of an optical characteristic changing material, which exerts an optical effect on the spectrum of light striking the core 30 in accordance with the amount of bending in a specific direction, this optical effect being different from the optical effect on the other detection targets 26. The detection target material 36 is of a soft material or elastic material, for example, a material with a low refractive index, resin, such as an acrylic resin, epoxy resin, silicone resin, or a fluororesin, a soft water glass, etc. In the meantime, the detection target material 36 may be formed to have a thickness substantially equal to the thickness of the clad, and a material like the jacket may be filled in that part on this detection target material 36, from which the jacket 34 and clad 32 were removed. Thereby, the light guide 24 may be configured to be capable of restoring to the original shape.

Besides, the jacket 34 and clad 32 are removed by a laser process, or by utilizing a photo process and an etching process. At this time, if a microscopic scar is caused on the core 30, light would leak, light to be guided would be lost, or the fragility to bending would increase. It is thus desirable to perform the process by a method that can prevent as much as possible a scar from occurring on the core 30.

As the optical characteristic changing material of which the detection target material 36 is composed, light absorbers having different light absorption spectra, as shown in FIG. 4, may be used in the respective detection targets 26. Specifically, in each detection target 26, light of a predetermined wavelength range is absorbed. Thus, the amount of bending of the detection target 26 can be calculated based on this light quantity by detecting the quantity of light of this wavelength.

Alternatively, the detection target material 36 may be composed of an optical characteristic changing material formed of a metal particle that absorbs light of a predetermined wavelength range. The optical characteristic changing material formed of the metal particle has a special spectral absorption spectrum different from the special spectral absorption spectrum that is inherent to the metal. For example, the optical characteristic changing material formed of the metal particle has a photoexcitation plasmon generation function that can excite a plasmon by light of at least one kind of light source. Specifically, the optical characteristic changing material is a metal nanoparticle having as an absorption spectrum a sum of a spectral absorption spectrum inherent to the metal and a special absorption spectrum by a surface plasmon effect. The photoexcitation plasmon generation function is constituted by any one of at least one kind of plasmon substance, nanosized substance, nanosized mineral, and nanosized metal. Here, the plasmon substance is a substance having a state in which free electrons vibrate collectively and behave as pseudo-particles. In addition, “nanosized” means “less than 1 μm”. The metal particle is, for instance, Au, Ag, Cu, Pt, etc., and is a dispersion medium. The shape of the metal particle is spherical, circular columnar, or polygonal columnar.

Even in the case of the same optical characteristic changing material, for example, the same metal particle, if at least one of the size, length, and thickness differs, the special spectral absorption spectrum of the photoexcitation plasmon generation function varies. For example, as the particle size becomes larger, the peak wavelength of light absorptance (absorption wavelength characteristic region) moves toward the long wavelength side. Accordingly, for the detection targets 26, there is a combination of optical characteristic changing materials that have different special spectral absorption spectra of the same metal element.

In addition, as regards the photoexcitation plasmon generation function, in the case of a different optical characteristic changing material, for example, a different metal particle, the special spectral absorption spectrum varies.

Moreover, it is also possible to use a composite optical characteristic changing material in which metal particles are mixed.

Accordingly, by using plural optical characteristic changing materials, for example, plural metal particles by differentiating at least one of their sizes, lengths, and thicknesses, detection target materials 36 having mutually different special spectral absorption spectra can be obtained. Many detection targets 26, each of which imparts an optical characteristic change that is different from the optical characteristic changes of other detection targets 26, can be formed.

Besides, the optical characteristic changing material may be, for example, an optical characteristic changing material including a multilayer dielectric film, an optical characteristic changing material including a fluorescent substance, or an optical characteristic changing material including a grating structure.

In the shape calculating apparatus 10 with the above-described configuration, light enters the light guide 24 from the light source 14 through the light branching element 18. The light that has entered is reflected by the reflection member 28 at the distal end of the light guide 24. The light that has reflected is received by the light detector 16 through the light branching element 18. The light received by the light detector 16 is the light that has passed through the detection targets 26 (first detection target 26-1, second detection target 26-2, . . . , n-th detection target 26-n) and varies in accordance with the curvature of the light guide 24. The quantity of light received by the light detector 16 and having the wavelength relating to each detection target 26 is delivered to the processor 22 as light quantity information (Dλn). Based on this light quantity information, the processor 22 calculates the curvature information.

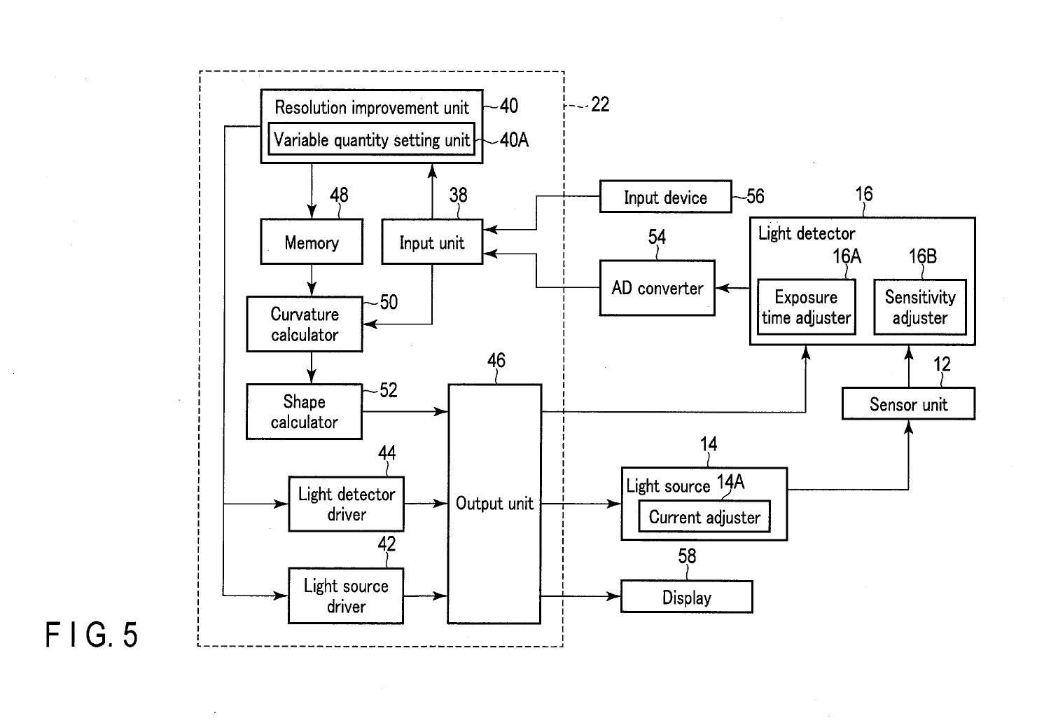

As illustrated in FIG. 5, the light source 14 can include a current adjuster 14A that changes the intensity of light that is emitted. In addition, the light detector 16 can include an exposure time adjuster 16A that changes the exposure time. Alternatively, the light detector 16 can include a sensitivity adjuster 16B that changes the sensitivity by changing the gain setting of a charge amplifier circuit (not shown) of the light detector 16. The details of these functions will be described later.

Besides, the processor 22 includes an input unit 38, a resolution improvement unit 40, a light source driver 42, a light detector driver 44, an output unit 46, a memory 48, a curvature calculator 50, and a shape calculator 52. The processor 22 can be composed by, for example, a computer.

The input unit 38 receives input data, which is delivered from the outside of the processor 22, and supplies, as needed, the input data to the resolution improvement unit 40 and curvature calculator 50. Specifically, a detection signal of each wavelength of the sensor unit 12, which is converted to digital data by an AD converter 54, is input to the input unit 38 from the light detector 16. Further, an exposure end signal is input to the input unit 38 from the light detector 16. Besides, a curvature computation start signal, a curvature computation end signal, sensor identification information, a signal relating to the setting of the curvature calculator 50, etc. are input to the input unit 38 from an input device 56. The input device 56 includes a switch or button for instructing the start/end of curvature computation. The input device 56 also includes a keyboard for setting up the kind of sensor unit 12 and setting up the curvature calculator 50 by inputting information to a menu or selection items displayed on a display 58. The input device 56 may further include a communication device that inputs information from the outside through a wired or wireless network.

The resolution improvement unit 40 performs the function of improving the resolution of light quantity information by changing the dynamic range of one of the intensity of light that is input to the sensor unit 12 and the electric signal that is generated by the light detector 16 based on light that is output from the sensor unit 12. The resolution improvement unit 40 includes a variable quantity setting unit 40A that changes any setting of the light intensity by the current adjuster 14A of the light source 14, the exposure time by the exposure time adjuster 16A of the light detector 16, and the gain setting of the charge amplifier circuit by the sensitivity adjuster 16B of the light detector 16, when the input unit 38 acquires the exposure end signal from the light detector 16. In the present embodiment, the variable quantity setting unit 40A changes this setting stepwise, thereby changing the dynamic range in a stepwise manner. In addition, the variable quantity setting unit 40A executes this stepwise change each time the input unit 38 acquires the exposure end signal from the light detector 16, thereby executing a sequential setting change.

Concretely, the variable quantity setting unit 40A transmitting a sequential order signal, which indicates a sequential order number of a setting among stepwise settings of X steps, to the light source driver 42 or light detector driver 44, so that the setting change by the variable quantity setting unit 40A is executed.

Specifically, when the light intensity is changed stepwise, the variable quantity setting unit 40A can transmit the sequential order signal to the light source driver 42. The light source driver 42 changes the information of the set light intensity based on the transmitted sequential order signal. In addition, the light source driver 42 transmits the information of the light intensity, which has been newly set by the change, to the current adjuster 14A of the light source 14 through the output unit 46. The current adjuster 14A drives the LD or the like by the driving current corresponding to the information of the light intensity from the light source driver 42, thus being able to adjust the intensity of light that is input to the sensor unit 12.

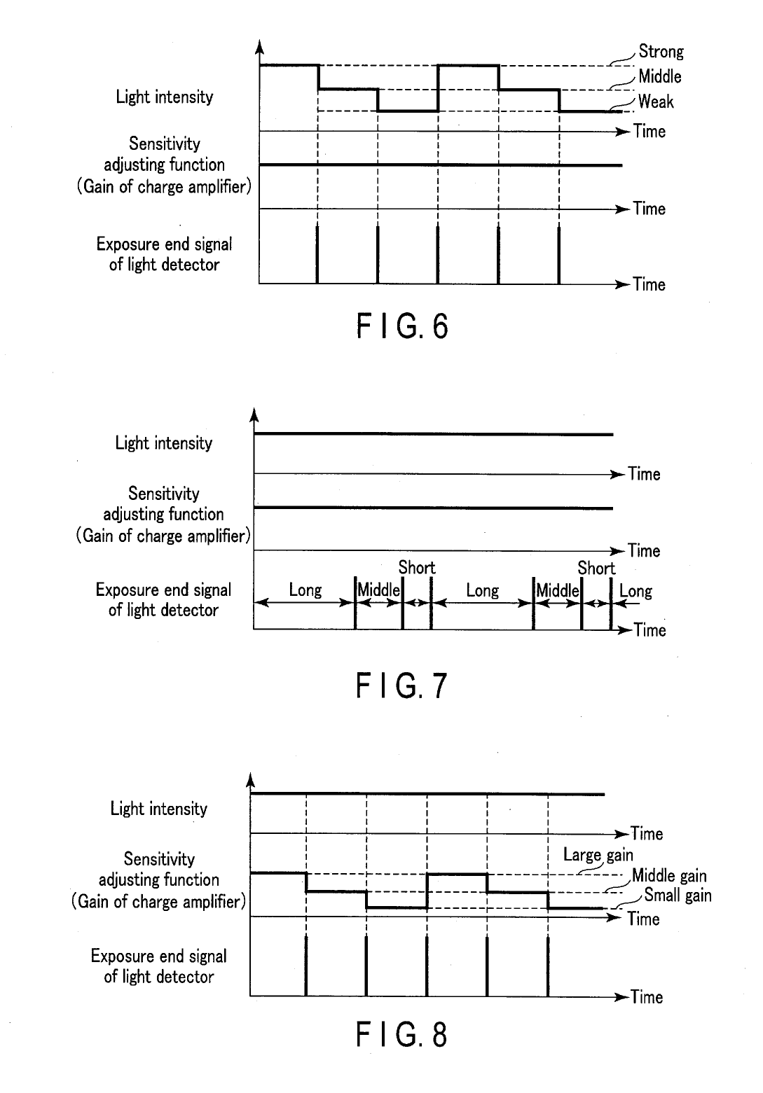

When the light intensity is changed stepwise by the current adjuster 14A of the light source 14, the variable quantity setting unit 40A successively transmits sequential order numbers to the light source driver 42. As illustrated in the time chart of FIG. 6, each time the input unit 38 receives an exposure end signal from the light detector 16, the light intensity of three steps (i.e. X=3) is sequentially changed in the order of “sequential order number 1: strong”→“sequential order number 2: middle”→“sequential order number 3: weak”→“sequential order number 1: strong”→, . . . . At this time, since the sequential order number is not transmitted to the light detector driver 44, the exposure time and the gain setting of the charge amplifier circuit are not adjusted. The exposure end signal, which the input unit 38 acquires from the light detector 16, is acquired at regular intervals. The sensitivity of the light detector 16 is constant, regardless of time.

When the exposure time is changed stepwise, the variable quantity setting unit 40A can transmit the sequential order signal to the light detector 44. The sequential order signal is associated with the exposure time so that the light detector driver 44 changes the information of the set exposure time based on the transmitted sequential order signal. In addition, the light detector driver 44 transmits the information of the exposure time, which has been newly set by the change, to the exposure time adjuster 16A of the light detector 16 through the output unit 46. The exposure time adjuster 16A detects the detection signal of each wavelength from the sensor unit 12, with the exposure time corresponding to the information of the exposure time from the light detector driver 44. Thereby, the exposure time adjuster 16A can adjust the electric signal that is generated by the light detector 16 based on the light that is output from the sensor unit 12.

Accordingly, when the exposure time is changed stepwise by the exposure time adjuster 16A of the light detector 16, the variable quantity setting unit 40A successively transmits sequential order signals to the light detector driver 44. As illustrated in the time chart of FIG. 7, the exposure time of three steps is sequentially changed in the order of “sequential order number 1: long”→“sequential order number 2: middle”→“sequential order number 3: short”→“sequential order number 1: long”→, . . . . Thereby, the exposure end signal, which the input unit 38 acquires from the light detector 16, is not acquired at regular intervals, but changes with time. At this time, since the sequential order number is not output to the light source driver 42, the intensity of light emitted by the light source 14 becomes constant, regardless of time. In addition, in the light detector driver 44, the gain setting of the charge amplifier circuit of the light detector 16 is not associated with the sequential order signals. Thus, the sensitivity of the light detector 16 is also constant, regardless of time.

Alternatively, when the gain setting of the charge amplifier circuit of the light detector 16 is changed stepwise, the variable quantity setting unit 40A can transmit sequential order signals to the light detector driver 44. The gain setting of the charge amplifier circuit is associated with the sequential order signals, so that the light detector driver 44 changes the set information of the gain setting of the charge amplifier circuit based on the transmitted sequential order signal. In addition, the light detector driver 44 outputs the information of the gain setting of the charge amplifier circuit, which has been newly set by the change, to the sensitivity adjuster 16B of the light detector 16 through the output unit 46. The sensitivity adjuster 16B detects the detection signal of each wavelength from the sensor unit 12, with the sensitivity corresponding to the information of the gain setting of the charge amplifier circuit from the light detector driver 44. Thereby, the sensitivity adjuster 16B can adjust the electric signal that is generated by the light detector 16 based on the light that is output from the sensor unit 12.

Accordingly, when the gain setting of the charge amplifier circuit is changed stepwise by the sensitivity adjuster 16B of the light detector 16, the variable quantity setting unit 40A successively sets sequential order signals to the light detector driver 44. As illustrated in the time chart of FIG. 8, each time the input unit 38 acquires the exposure end signal from the light detector 16, the sensitivity of three steps is sequentially changed in the order of “sequential order number 1: large gain”→“sequential order number 2: middle gain”→“sequential order number 3: small gain”→“sequential order number 1: large gain”→, . . . . At this time, in the light detector driver 44, the exposure time is not associated with the sequential order signals. Thus, the exposure time is not adjusted, and the exposure end signal, which the input unit 38 acquires from the light detector 16, is acquired at regular intervals. In addition, since the sequential order signal is not output to the light source driver 42, the intensity of light emitted by the light source 14 becomes constant, regardless of time.

In the meantime, the association of the exposure time of the light detector 16 or the sensitivity of the light detector 16 in the light detector driver 44 with the sequential order signals is performed in advance at a time of factory shipment or the like. Alternatively, the association may be made changeable based on the sensor identification information that is input to the input unit 38 from the input device 56.

As to whether the variable quantity setting unit 40A transmits the sequential order signals to the light source driver 42 or to the light detector driver 44, this selection may be made in advance at a time of factory shipment or the like, or may be made based on the sensor identification information that is input to the input unit 38 from the input device 56.

Needless to say, the number X of steps of the sequential change by the variable quantity setting unit 40A is not limited to three (X=3). The number X may be two (X=2) or may be four or more (X≥4).

The memory 48 prestores curvature characteristic information corresponding to various settings of the light detector 16 and light source 14, with respect to each kind of the sensor unit 12 that is usable.

The curvature calculator 50 calculates the curvature information of each detection target 26 (first detection target 26-1, second detection target 26-2, n-th detection target 26-n) of the sensor unit 12 based on the optimal light quantity information (to be described later in detail) of detection signals corresponding to the adjustment, which the input unit 38 acquires, and based on the curvature characteristic information that corresponds to the various settings of the light detector 16 and light source 14, which is stored in the memory 48, corresponding to the sensor identification information that is input to the input unit 38 from the input device 56. The curvature calculator 50 transmits the calculated curvature information of each detection target 26 to the shape calculator 52.

The shape calculator 52 converts the curvature information of each detection target 26 to shape information of a structure body such as the insertion section of the endoscope. The shape calculator 52 transmits this shape information of the structure body to the display 58 through the output unit 46.

The display 58 displays the shape information of the structure body.

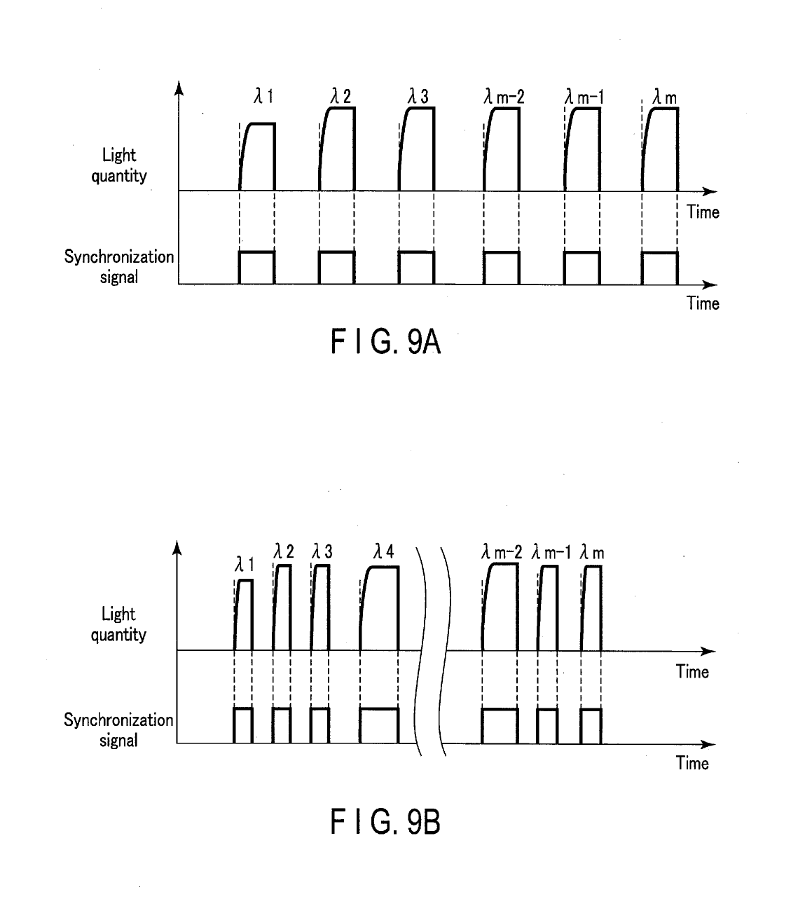

Besides, as shown in FIG. 9A, as the light detector 16, a light detector of such a type that the wavelength of detection, i.e. the wavelength of exposure, changes in accordance with a synchronization signal may be used. However, if the light detector 16 of this type is used, when the exposure time is changed stepwise by the exposure time adjuster 16A, it is necessary to adjust the cycle (frequency) of the synchronization signal so that all wavelengths (λ1-λm: m>n) are exposed within the changed exposure time.

In addition, if the cycle (frequency) of the synchronization signal is simply adjusted, the exposure time for each wavelength becomes short when the exposure time is set to “short”, and there is concern that the AD converter 54 cannot acquire all of high-precision data. On the other hand, the wavelengths that are used for the detection targets 26 of the sensor unit 12, that is, the wavelengths that are used for the curvature calculation, are only a part of all wavelengths (λ1-λm), for example, λ4-λm−2 (in this case, n=m−5). It should suffice if the detection signals of the wavelengths that are used for the curvature calculation are obtained with high precision. Thus, as shown in FIG. 9B, it is desirable to use such synchronization signals that the cycle is longer (the frequency is lower) in the wavelength corresponding to each detection target 26, and the cycle is shorter (the frequency is higher) in the wavelength that is not used in the curvature calculation.

Thus, when the exposure time information is set to “short”, the light detector driver 44 can also change the setting information of the synchronization signal so that the synchronization signal that is varied in accordance with the wavelength is supplied to the light detector 16 from the output unit 46.

Besides, the varying of the synchronization signal may be implemented not only when the exposure time information is set to “short”, but may also be implemented at all times when the sequential change of X steps is executed as in the present embodiment. If the above-described sequential change, for example, the change of three steps, is executed, the acquisition of the light quantity information, which the processor 22 uses for the curvature calculation, requires three times the period, compared to the case in which the change is not executed. By varying the synchronization signal in accordance with the wavelength range that is used, the total light quantity information acquisition time that is necessary for one-time curvature calculation can be decreased.

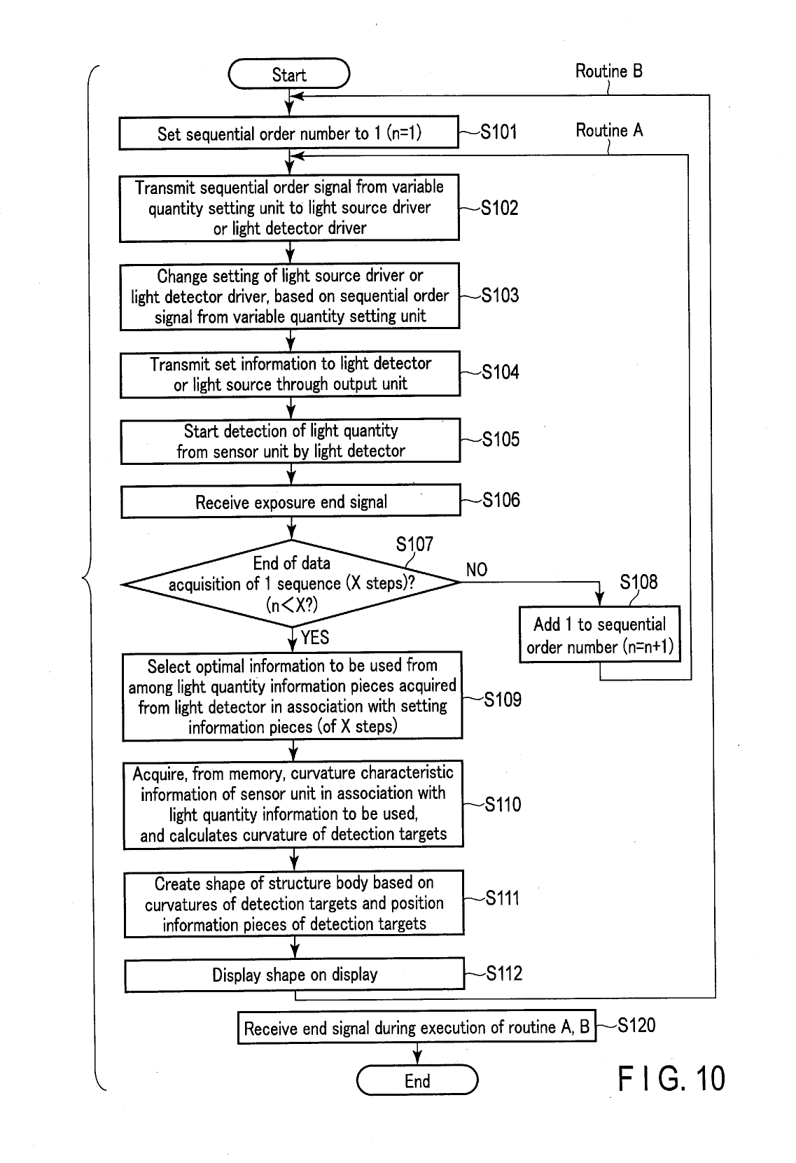

Hereinafter, the operation of the processor 22 of the shape calculating apparatus 10 according to the first embodiment will be further described with reference to a flowchart of FIG. 10.

If the input unit 38 receives a curvature computation start signal from the input device 56, the operation of this flowchart is started. To begin with, the resolution improvement unit 40 sets the sequential order number n, which the variable quantity setting unit 40A is to transmit, to 1, that is, executes the initial setting of n=1 (step S101).

Then, the resolution improvement unit 40 transmits, by the variable quantity setting unit 40A, a sequential order signal to the light source driver 42 or light detector driver 44 (step S102)

Based on the sequential order signal from the variable quantity setting unit 40A, the light source driver 42 or light detector driver 44 that received the sequential order signal changes the setting of the light source driver 42 or light detector driver 44 (step S103), and transmits the set information to the light source 14 or light detector 16 through the output unit 46 (step S104). Thereby, the setting of the light intensity, exposure time, or sensitivity that corresponds to the sequential order number is changed.

After the change of the setting, the emission of light from the light source 14 is started, and the light detector 16 starts the light quantity detection of each wavelength of light from the sensor unit 12 (step S105). The detected light quantity information is input to the input unit 38 through the AD converter 54. The input light quantity information is temporarily stored in a memory (not shown) that is constituted in the input unit 38. Alternatively, the light quantity information may be supplied to the memory 48 from the input unit 38 and may be stored in the memory 48.

If the light detector 16 completes detection of light quantities of all wavelengths (λ1-λm), the light detector 16 outputs an exposure end signal. If the input unit 38 receives the exposure end signal from the light detector 16 (step S106), the resolution improvement unit 40 determines whether the data acquisition of one sequence (X steps) has been finished or not, that is, whether n=X (step S107).

Here, if it is determined that the data acquisition of one sequence is not finished, that is, if n<X, then 1 is added to the sequential order number, that is, the sequential order number is set to n=n+1 (step S108). Then, the operation returns to the process of step S102.

In this manner, a routine A of step S102 to step S108 is repeated. Thereby, the light quantity information is detected with the light intensity, exposure time, or sensitivity that has been set by the setting information of X steps.

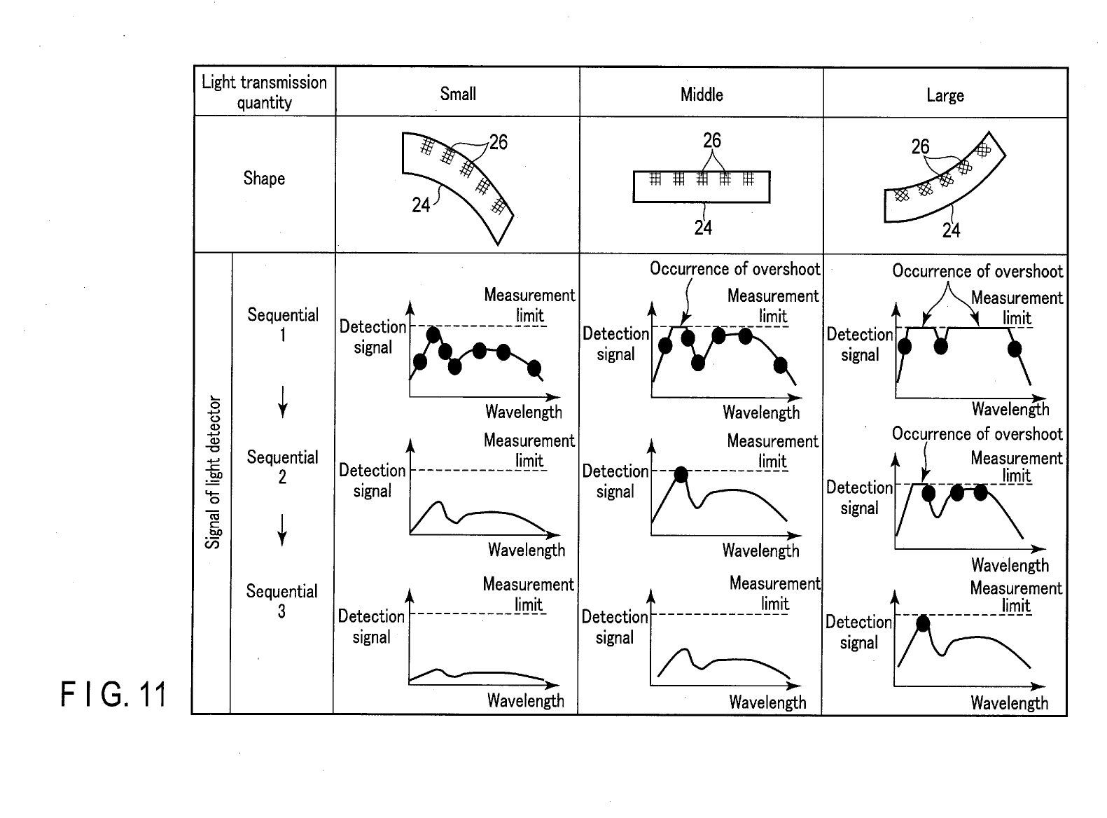

For example, as shown in FIG. 11, in Sequential 1 in which n=1, the setting information of the light intensity, exposure time, or sensitivity is set so that the largest value of the detection signal of the light detector 16 is a value that is approximately a measurement limit of the light detector 16, when the light guide 24 is bent toward the side opposite to the side where the detection target 26 is provided, as shown in FIG. 3B, with the light transmission quantity being small. Accordingly, even in this bent state, the quantity of light with the wavelength corresponding to each detection target 26 can be detected, and the light quantity information of all detection targets 26 can be acquired with high resolution. Incidentally, in FIG. 11, a black circle indicates light quantity information acquired in association with each detection target 26.

However, with this setting information, an overshoot portion exceeding the measurement limit of the light detector 16 occurs in the detection signal of the light detector 16 when the light guide 24 is not bent, as shown in FIG. 3A, with the light transmission quantity being middle, or when the light guide 24 is bent toward the side where the detection target 26 is provided, as shown in FIG. 3C, with the light transmission quantity being large. In this overshoot portion, the light quantity information cannot be acquired.

Thus, in the next Sequential 2 in which n=2, the setting information of the light intensity, exposure time, or sensitivity is set so that the largest value of the detection signal of the light detector 16 is approximately the measurement limit of the light detector 16, when the light guide 24 is not bent, as shown in FIG. 3A, with the light transmission quantity being middle. Thereby, the light quantity information that could not be acquired due to the overshoot in the setting of Sequential 1 can be acquired with high resolution.

However, even with the setting information of Sequential 2, an overshoot portion still occurs when the light guide 24 is bent toward the side where the detection target 26 is provided, as shown in FIG. 3C, with the light transmission quantity being large.

Thus, in the next Sequential 3 in which n=3, the setting information of the light intensity, exposure time, or sensitivity is set so that the largest value of the detection signal of the light detector 16 is approximately the measurement limit of the light detector 16, when the light guide 24 is bent toward the side where the detection target 26 is provided, as shown in FIG. 3C, with the light transmission quantity being large. Thereby, the light quantity information that could not be acquired due to the overshoot in the settings of Sequential 1 and Sequential 2 can be acquired with high resolution.

In this manner, if the data acquisition of one sequence of sequences 1 to X (X=3 in the example of FIG. 11) is finished, n=X is determined in step S107. Then, the curvature calculator 50 selects the optimal light quantity information for use in the curvature calculation, from among the light quantity information pieces acquired from the light detector 16 in association with the plural setting information pieces (of X steps) (step S109).

Specifically, if the light quantity information pieces of all detection targets 26 are successfully acquired in Sequential 1, the curvature calculator 50 selects these information pieces as the optimal light quantity information for use in the curvature calculation. On the other hand, if there is a detection target 26 the light quantity information of which could not be acquired due to the overshoot, the curvature calculator 50 selects the light quantity information acquired in Sequential 2 with respect to the light quantity information of the detection target 26. Moreover, if there is a the detection target 26 the light quantity information of which could not be acquired in Sequential 2 due to the overshoot, the curvature calculator 50 selects the light quantity information acquired in Sequential 3 with respect to the light quantity information of the detection target 26. In this manner, the curvature calculator 50 selects the optimal (largest) light quantity information with no overshoot. Alternatively, it is also possible to determine in advance which of the detection signals of Sequential 1 to Sequential 3 is to be selected as the light quantity information with respect to each wavelength.

In addition, the curvature calculator 50 acquires from the memory 48 the curvature characteristic information of the sensor unit 12 in association with these selected light quantity information pieces to be used, and then calculates the curvatures of the detection targets 26 (step S110). Specifically, since the curvature characteristic information differs depending on which of Sequential 1 to Sequential 3 is associated with each light quantity information piece to be used, the curvature characteristic information corresponding to each light quantity information piece is acquired, and the curvatures of the detection targets 26, which each correspond to the light quantity information pieces, are calculated.

The shape calculator 52 creates the shape of the structure body based on the curvatures of the detection targets 26 that have been calculated by the curvature calculator 50 and the position information pieces of the detection targets 26 that is prior information (step S111). Then, the shape calculator 52 displays the created shape of the structure body on the display 58 through the output unit 46 (step S112).

Thereafter, the operation from step S101 is repeated.

In this manner, a routine B of step S101 to step S112 is repeated. Thereby, the shape of the structure body corresponding to the displacement of the structure body can be updated and displayed on the display 58.

If the input unit 38 receives a curvature computation end signal from the input device 56 while the above routine A or routine B is being executed (step S120), the process of this flowchart is terminated.

As described above, the shape calculating apparatus 10 according to the first embodiment includes the light detector 16 configured to detect the light quantity information that is the relationship between the wavelengths and light quantities acquired by using the sensor unit 12 that is configured such that the light quantity detected with respect to the wavelength corresponding to each of the detection targets 26 varies in accordance with the shape of each of the detection targets 26; the curvature calculator 50 configured to execute a calculation relating to the shape of each detection target 26 based on the light quantity information; and the setting change unit (resolution improvement function) configured to change the dynamic range of either the intensity of light that is input to the sensor unit 12 or the detection signal of the light detector 16 that is the electric signal generated by the light detector 16 based on the light that is output from the sensor unit 12. Here, the setting change unit includes, in addition to the variable quantity setting unit 40A of the resolution improvement unit 40, any one of a set of the light source driver 42 and the current adjuster 14A of the light source 14; a set of the light detector driver 44 and the exposure time adjuster 16A of the light detector 16; and a set of the light detector driver 44 and the sensitivity adjuster 16B of the light detector 16.

The shape calculating apparatus 10 changes the dynamic range of either the intensity of light that is input to the sensor unit 12 or the electric signal generated by the light detector 16 based on the light that is output from the sensor unit 12. Thereby, the shape calculating apparatus 10 can acquire, with high precision, the light quantity information that is the relationship between the wavelengths and light quantities from the sensor unit 12 that includes the detection targets 26. Therefore, the shape of each detection target 26 can exactly be calculated.

Specifically, the variable quantity setting unit 40A of the resolution improvement unit 40, the light detector driver 44, and the exposure time adjuster 16A of the light detector 16 can change the dynamic range of the detection signal of the light detector 16 by changing the exposure time of the light detector 16.

In the meantime, the variable quantity setting unit 40A and light detector driver 44 may change the dynamic range of the detection signal of the light detector 16 by changing the frequency of the synchronization signal relating to the detection of the light detector 16.

Alternatively, the variable quantity setting unit 40A, the light detector driver 44, and the sensitivity adjuster 16B of the light detector 16 can change the dynamic range of the detection signal of the light detector 16 by changing the detection sensitivity of the light detector 16.

Besides, the variable quantity setting unit 40A, the light source driver 42, and the current adjuster 14A of the light source 14 can change the dynamic range of the intensity of light that is input to the sensor unit, by changing the intensity of light that is input to the sensor unit 12.

The shape calculating apparatus 10 can further include the input device 56 functioning as an instruction unit configured to a method to be used among these methods of changing the dynamic range.

Furthermore, the shape calculating apparatus 10 can execute the change of the dynamic range stepwise, and can sequentially execute this stepwise change.

Besides, the shape calculating apparatus 10 can further include the light source 14 configured to emit light and the sensor unit 12. Here, the sensor unit 12 includes the light guide 24 that is a light guide configured to guide light emitted from the light source 14; and the detection targets 26 including the respective detection target materials 36 that are provided in the light guide 24 and composed of optical characteristic change materials and that exert mutually different effects on the spectrum of light that is guided by the light guide 24. In addition, the light detector 16 detects the light that is guided by the light guide 24 and that is affected by the detection target materials 36 to output the light quantity information.

### Second Embodiment

Next, a second embodiment of the present invention will be described. Here, different points from the above-described first embodiment will be described, and the same parts are denoted by like reference numerals and a description thereof is omitted.

In the shape calculating apparatus 10 according to the first embodiment, the setting of the light intensity of the light source 14, the exposure time of the light detector 16, or the sensitivity of the light detector 16 is sequentially changed in accordance with the exposure end signal so that the magnitude of the detection signal of the light detector 16 changes stepwise in accordance with the exposure end signal.

By contrast, in a shape calculating apparatus 10 according to the second embodiment, the setting of the light intensity of the light source 14, the exposure time of the light detector 16, or the sensitivity of the light detector 16 is changed after determining the state in magnitude of the detection signal of the light detector 16.

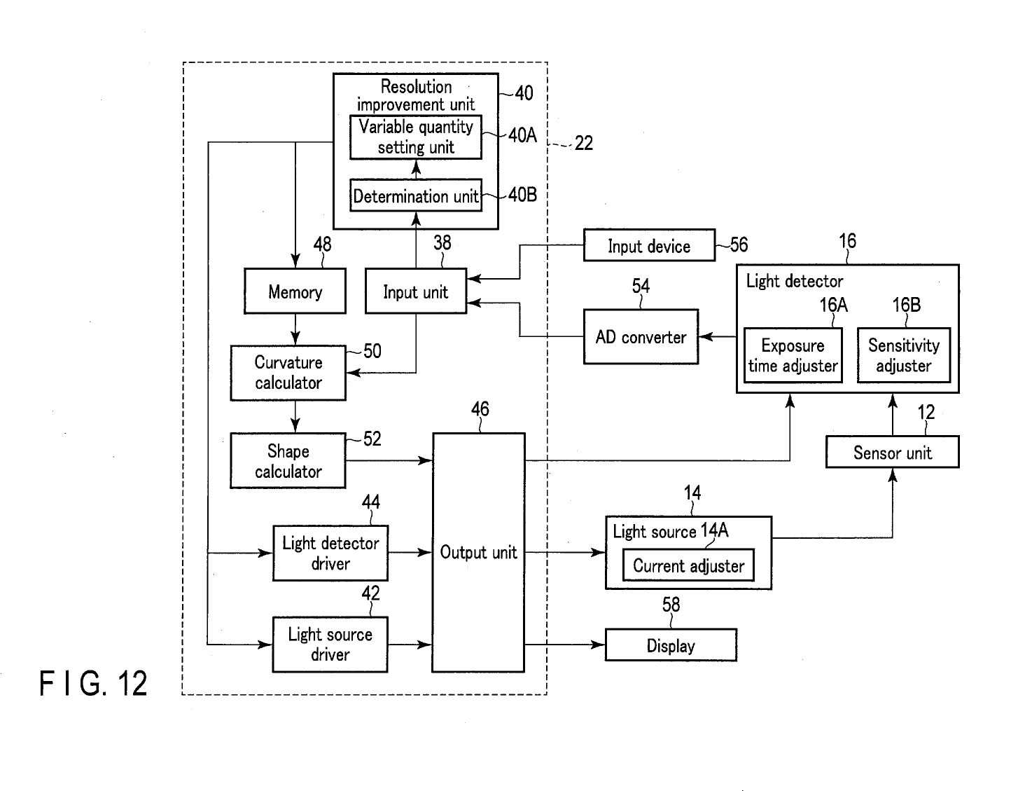

As illustrated in FIG. 12, in the shape calculating apparatus 10 of this embodiment, the resolution improvement unit 40 of the processor 22 includes, in addition to the variable quantity setting unit 40A, a determination unit 40B configured to determine whether the change of the dynamic range is executed or not; the variable quantity setting unit 40A operates based on the determination by the determination unit 40B. Here, the determination unit 40B compares the detection signal of the light detector 16 with a threshold (lower-limit threshold) relating to the lower limit of detection and a threshold (upper-limit threshold) relating to the upper limit of detection. Thereby, when the determination unit 40B has determined that the detection signal is not in the detectable range, the determination unit 40B determines that the dynamic range is to be changed.

The upper-limit threshold and lower-limit threshold for use in the determination unit 40B are prestored in the memory 48. Alternatively, the upper-limit threshold and lower-limit threshold may be input from the input device 56 to be stored in the memory 48. Specifically, the input device 56 can be used as an instruction unit configured to instruct a change of the information relating to the determination in the determination unit 40B.

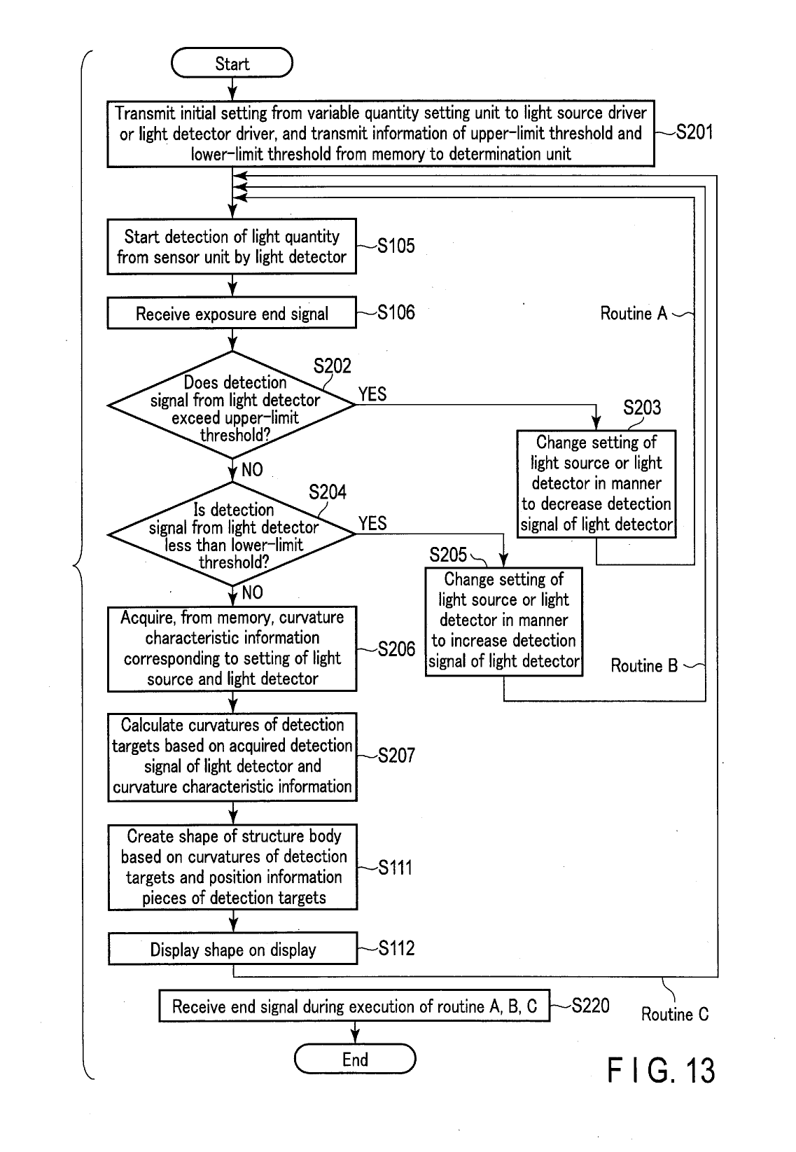

Hereinafter, the operation of the processor 22 of the shape calculating apparatus 10 according to the second embodiment will be described with reference to a flowchart of FIG. 13.

If the input unit 38 receives a curvature computation start signal from the input device 56, the operation of this flowchart is started. To begin with, the resolution improvement unit 40 transmits the initial setting from the variable quantity setting unit 40A to the light source driver 42 or light detector driver 44, and the resolution improvement unit 40 reads out the information of the upper-limit threshold and lower-limit threshold from the memory 48, and then sends the information to the determination unit 40B (step S201). In accordance with this initial setting from the variable quantity setting unit 40A, the light source driver 42 or light detector driver 44 changes the setting of the light source driver 42 or light detector driver 44, and then transmits the set information to the light source 14 or light detector 16 through the output unit 46. Thereby, the setting of the light intensity, exposure time, or sensitivity is set in the initial state. The initial set value of the light intensity, exposure time, or sensitivity is not particularly limited. For example, the sequential order number, which instructs the setting of Sequential 2 in the first embodiment, is transmitted from the variable quantity setting unit 40A to the light source driver 42 or light detector driver 44, and thereby the setting of Sequential 2 can be executed. Alternatively, by directly transmitting the setting information itself of the light intensity, exposure time, or sensitivity from the variable quantity setting unit 40A to the light source driver 42 or light detector driver 44, the setting change according to this setting information may be executed.

With the above initial setting being executed, the emission of light from the light source 14 is started, and the light detector 16 starts the light quantity detection of each wavelength of light from the sensor unit 12 (step S105). The detected light quantity information is input to the input unit 38 through the AD converter 54 and then stored in a memory (not shown) that is constituted in the input unit 38 or in the memory 48.

If the light detector 16 completes detection of light quantities of all wavelengths (λ1-λm), the light detector 16 outputs an exposure end signal. If the input unit 38 receives the exposure end signal from the light detector 16 (step S106), the determination unit 40B of the resolution improvement unit 40 determines whether the detection signal from the light detector 16 exceeds the upper-limit threshold or not (step S202). In the meantime, it is preferable that this upper-limit threshold is slightly less than the measurement limit of the light detector 16. In addition, the determination by the determination unit 40B may be executed with respect to all wavelengths of the detection signal of the light detector 16 or may be executed with respect to one specific wavelength or plural specific wavelengths that are designated in advance.

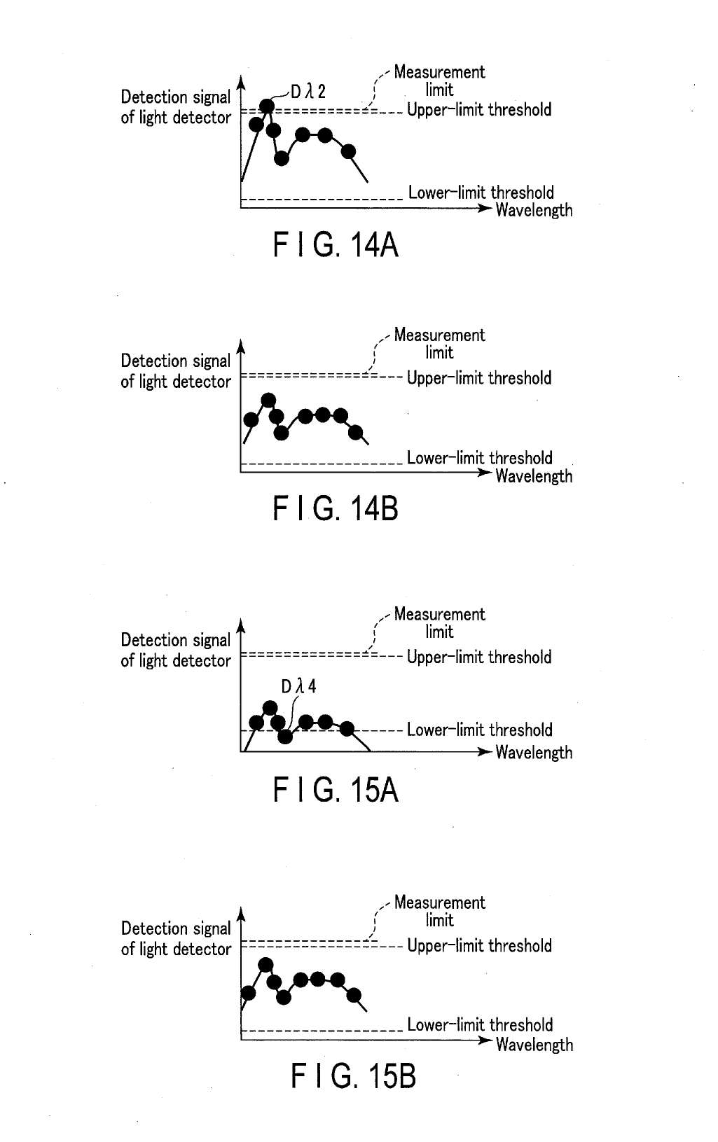

For example, as shown in FIG. 14A, if one (e.g. light quantity information Dλ2) of light quantity information pieces used in the curvature calculation exceeds the upper-limit threshold, that is, if the light intensity of one wavelength used in the curvature calculation in the detection signal of the light detector 16 exceeds the upper-limit threshold, the determination unit 40B outputs information indicative of this to the variable quantity setting unit 40A.

Upon receiving the information indicating that one of the light quantity information pieces used in the curvature calculation exceeds the upper-limit value, the variable quantity setting unit 40A changes the setting of the light source 14 or light detector 16 so that the detection signal of the light detector 16 decreases as shown in FIG. 14B (step S203). Specifically, the variable quantity setting unit 40A transmits, to the light source driver 42 or light detector driver 44, the sequential order number or setting information for changing the setting of the current adjuster 14A of the light source 14, the exposure time adjuster 16A of the light detector 16, or the sensitivity adjuster 16B of the light detector 16 so that the detection signal of the light detector 16 decreases. Then, the operation returns to the process of step S105.

Besides, in step S203, the setting of the light source 14 or light detector 16 may not only be changed, but the upper-limit threshold that is the reference for determination of the determination unit 40B may also be changed. Specifically, the upper-limit threshold can be changed to an optimal upper-limit threshold for the detection signal of the light detector 16 after the change of the setting.

In this manner, a routine A of step S105, step S106, step S202, and step S203 can be repeated. Specifically, if the detection signal exceeds the upper-limit threshold even after the setting change, the setting of the current adjuster 14A of the light source 14, the exposure time adjuster 16A of the light detector 16, or the sensitivity adjuster 16B of the light detector 16 is changed through the light source 14 or light detector driver 44 so that the detection signal of the light detector 16 further decreases. In this way, the setting change of the current adjuster 14A of the light source 14, the exposure time adjuster 16A of the light detector 16, or the sensitivity adjuster 16B of the light detector 16 can be executed stepwise so that the detection signal of the light detector 16 can be acquired with the optimal setting.

On the other hand, in step S202, if the determination unit 40B determines that the detection signal from the light detector 16 does not exceed the upper-limit threshold, the determination unit 40B further determines whether the detection signal from the light detector 16 is less than the lower-limit threshold (step S204). Like the determination relating to the upper-limit value, this determination by the determination unit 40B may be executed with respect to all wavelengths of the detection signal of the light detector 16 or may be executed with respect to one specific wavelength or plural specific wavelengths that are designated in advance.

For example, as shown in FIG. 15A, if one (e.g. light quantity information Dλ4) of light quantity information pieces used in the curvature calculation decreases below the lower-limit threshold, the determination unit 40B outputs information indicative of this to the variable quantity setting unit 40A. Upon receiving this information, the variable quantity setting unit 40A changes the setting of the light source 14 or light detector 16 so that the detection signal of the light detector 16 increases as shown in FIG. 15B (step S205). Specifically, the variable quantity setting unit 40A transmits, to the light source driver 42 or light detector driver 44, the sequential order number or setting information for changing the setting of the current adjuster 14A of the light source 14, the exposure time adjuster 16A of the light detector 16, or the sensitivity adjuster 16B of the light detector 16 so that the detection signal of the light detector 16 increases. Thereby, for example, in such a case that the detection signal of the light detector 16 decreased below the lower-limit threshold as a result of the setting change in step S203 that decreased the detection signal of the light detector 16, the setting of the light source 14 or light detector 16 can be changed backward by one step. Then, the operation returns to the process of step S105.

Besides, in step S205, the setting of the light source 14 or light detector 16 may not only be changed, but the lower-limit threshold that is the reference for determination of the determination unit 40B may also be changed. Specifically, the lower-limit threshold can be changed to an optimal lower-limit threshold for the detection signal of the light detector 16 after the change of the setting.

In this manner, a routine B of step S105, step S106, step S202, step S204, and step S205 can be repeated. Specifically, if the detection signal is less than the lower-limit threshold even after the setting change, the setting of the current adjuster 14A of the light source 14, the exposure time adjuster 16A of the light detector 16, or the sensitivity adjuster 16B of the light detector 16 is changed through the light source driver 42 or light detector driver 44 so that the detection signal of the light detector 16 further increases. In this way, the setting change of the current adjuster 14A of the light source 14, the exposure time adjuster 16A of the light detector 16, or the sensitivity adjuster 16B of the light detector 16 can be executed stepwise so that the detection signal of the light detector 16 can be acquired with the optimal setting.

If the determination unit 40B determines, in step S202, that the detection signal from the light detector 16 does not exceed the upper-limit threshold and if the determination unit 40B determines, in step S204, that the detection signal from the light detector 16 is not less than the lower-limit threshold, the curvature calculator 50 acquires, from the memory 48, the curvature characteristic information corresponding to the setting of the light source 14 and light detector 16 (step S206). Specifically, the curvature calculator 50 acquires, from the memory 48, the curvature characteristic information based on the setting information of the current adjuster 14A of the light source 14, the exposure time adjuster 16A of the light detector 16, or the sensitivity adjuster 16B of the light detector 16 from the variable quantity setting unit 40A of the resolution improvement unit 40. In addition, based on the acquired detection signal of the light detector 16 and this curvature characteristic information, the curvature calculator 50 calculates the curvature of each detection target 26 (step S207).

The process of creating the shape of the structure body in subsequent step S111 and the process of displaying the shape in step S112 are the same as in the first embodiment.

Thereafter, the operation from step S105 is repeated.

In this manner, a routine C of step S105 to step S112 is repeated. Thereby, the shape of the structure body corresponding to the displacement of the structure body can be updated and displayed on the display 58.

If the input unit 38 receives a curvature computation end signal from the input device 56 while the above routine A, routine B, or routine C is being executed (step S220), the process of this flowchart is terminated.

### Third Embodiment

Next, a third embodiment of the present invention will be described. Here, different points from the above-described first embodiment will be described, and the same parts are denoted by like reference numerals and a description thereof is omitted.

In the shape calculating apparatus 10 according to the first embodiment, any one of the light intensity of light that is input to the sensor unit 12, the exposure time of the light detector 16, and the detection sensitivity of the light detector 16 is changed. Thereby, the dynamic range of either the intensity of light that is input to the sensor unit 12 or the electric signal generated by the light detector 16 based on the light output from the sensor unit 12 is changed. Specifically, the first embodiment is characterized by the intervention of the resolution improvement unit 40 before or when the light quantity is converted to the electric signal in the light detector 16.

By contrast, a shape calculating apparatus 10 according to the third embodiment is characterized by the intervention of the resolution improvement unit 40 after the light quantity has been converted to the electric signal. Specifically, by changing the range of the detection signal from the light detector 16, the dynamic range of the electric signal that is generated by the light detector 16 based on the light output from the sensor unit 12 is changed. To be more specific, a change relating to digital conversion of the electric signal is executed.

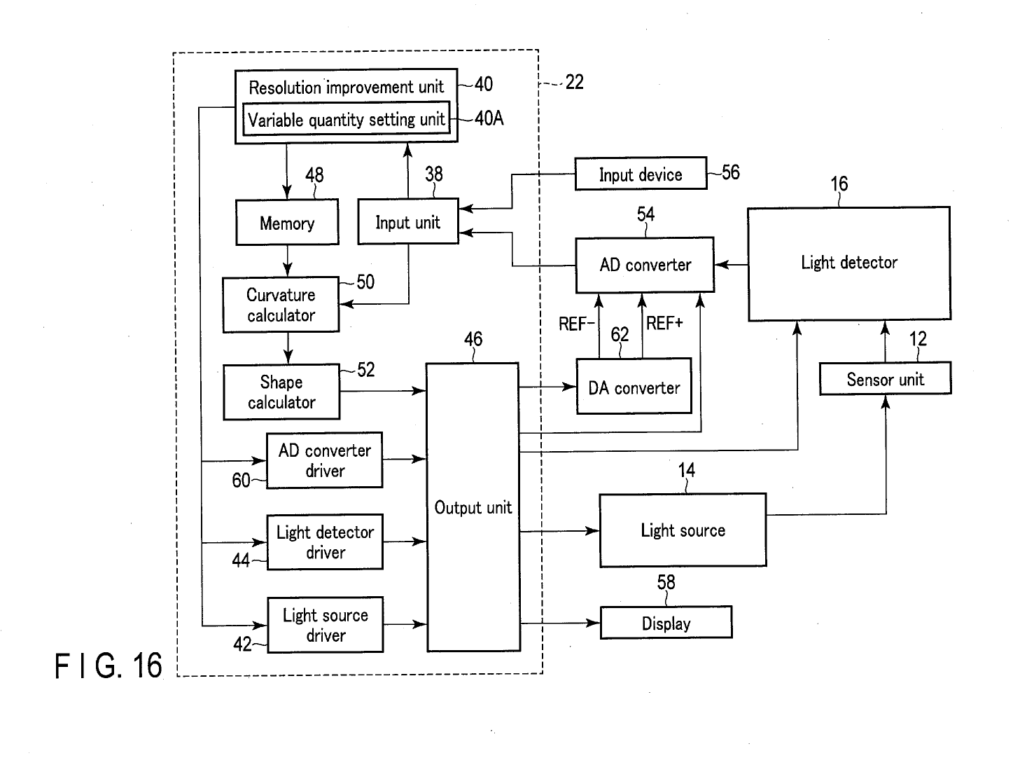

Thus, as illustrated in-FIG. 16, the shape calculating apparatus 10 of this embodiment further includes an AD converter driver 60 in the processor 22, which outputs reference voltage data indicative of a reference voltage of a −side and a reference voltage of a +side of the AD converter 54 that converts the light quantity information from the light detector 16 to digital data; and a DA converter 62 that converts the reference voltage data to a −side reference voltage REF− and a +side reference voltage REF+ to apply the −side reference voltage REF− and +side reference voltage REF+ to the AD converter 54. The variable quantity setting unit 40A of the resolution improvement unit 40 transmits, to the AD converter driver 60, an instruction value as to how to set the above-described reference voltage data. The AD converter 54 executes digital conversion of the light quantity information in the range of between the applied −side reference voltage REF− and +side reference voltage REF+.

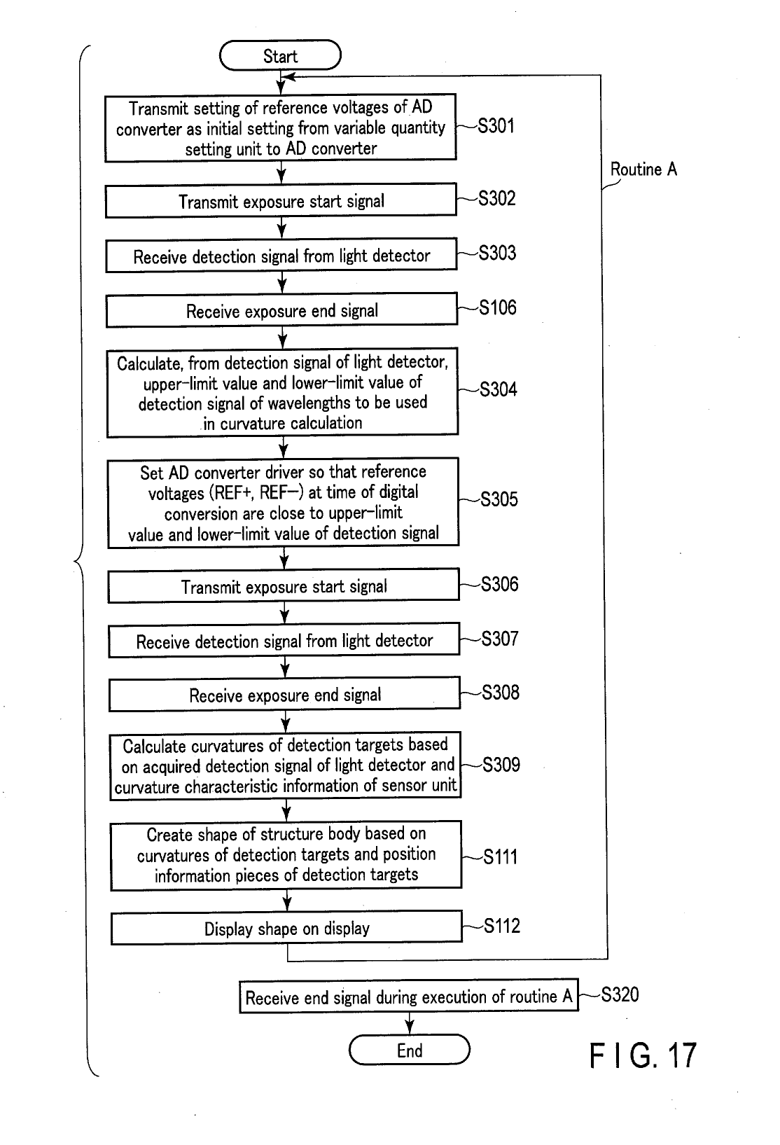

Hereinafter, the operation of the processor 22 of the shape calculating apparatus 10 according to the third embodiment will be described with reference to a flowchart of FIG. 17.

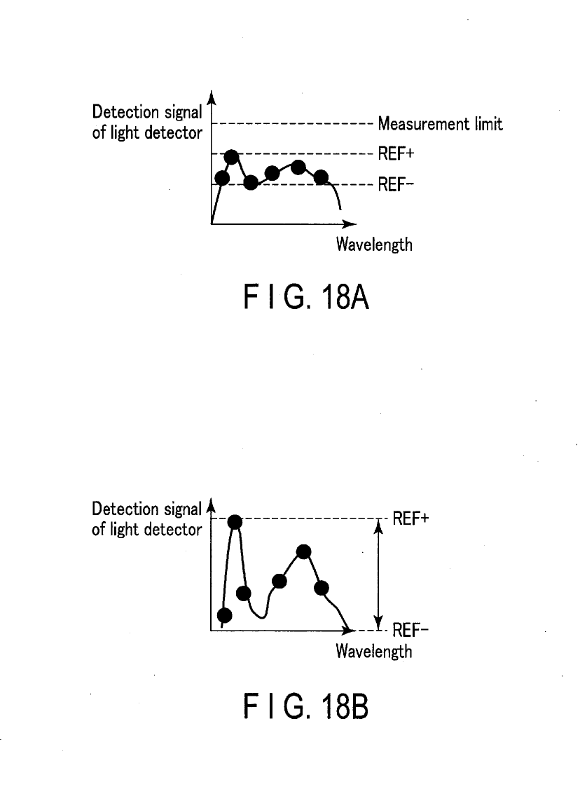

If the input unit 38 receives a curvature computation start signal from the input device 56, the operation of this flowchart is started. To begin with, the resolution improvement unit 40 transmits, from the variable quantity setting unit 40A, the setting of the reference voltages of the AD converter 54 as the initial setting to the AD converter 54 (step S301). Specifically, as shown in FIG. 18A, such an instruction value that a range from the measurement limit of the detection signal of the light detector 16 to GND is X bits that are a conversion bit number of the AD converter 54 is transmitted as the initial setting from the variable quantity setting unit 40A to the AD converter driver 60. The AD converter driver 60 transmits the received instruction value to the DA converter 62 through the output unit 46. The DA converter 62 applies the designated voltage of GND as the −side reference voltage REF− and the voltage of the measurement limit as the +side reference voltage REF+.

Then, the resolution improvement unit 40 causes the light source driver 42 to drive the light source 14 through the output unit 46, and causes the light detector driver 44 to transmit the exposure start signal to the light detector 16 through the output unit 46, thereby driving the light detector 16 (step S302). Thereby, the light detector 16 starts light quantity detection of each wavelength of the light from the sensor unit 12.

The input unit 38 receives, from the light detector 16, the detection signal that has been converted to the digital data by the AD converter 54, and the input unit 38 then stores the detection signal in the memory (not shown) constituted in the input unit 38 or in the memory 48 (step S303).

If the light detector 16 completes detection of light quantities of all wavelengths (λ1-λm), the light detector 16 outputs an exposure end signal. If the input unit 38 receives the exposure end signal from the light detector 16 (step S106), the variable quantity setting unit 40A of the resolution improvement unit 40 calculates, from the detection signal of the light detector 16, the upper-limit value and lower-limit value of the detection signal of the wavelengths to be used for the curvature calculation (step S304). For example, in the example of FIG. 18A, the value of the light quantity information Dλ2 of the detection signal of the light detector 16 is calculated as the upper-limit value of the detection signal, and the value of the light quantity information Dλ3 is calculated as the lower-limit value of the detection signal.

Then, the variable quantity setting unit 40A sets the AD converter driver 60 so that the reference voltages REF+ and REF− at the time of digital conversion are close to the upper-limit value and lower-limit value of the detection signal (step S305). Specifically, the variable quantity setting unit 40A sends the instruction value to the AD converter driver 60 such that the values in the neighborhoods of the calculated largest value and smallest value of the detection signal are the +side reference voltage REF+ and −side reference voltage REF−. The AD converter driver 60 transmits the received instruction value to the DA converter 62 through the output unit 46. The DA converter 62 applies voltages of the designated −side reference voltage REF− and +side reference voltage REF+ to the AD converter 54.

Thereafter, the resolution improvement unit 40 causes the light source driver 42 to drive the light source 14 through the output unit 46, and causes the light detector driver 44 to transmit the exposure start signal to the light detector 16 through the output unit 46, thereby driving the light detector 16 (step S306). Thereby, the light detector 16 starts light quantity detection of each wavelength of the light from the sensor unit 12.

The input unit 38 receives, from the light detector 16, the detection signal that has been converted to the digital data by the AD converter 54, and the input unit 38 stores the detection signal in the memory (not shown) constituted in the input unit 38 or in the memory 48 (step S307). At this time, as shown in FIG. 18A, the AD converter 54 executes digital conversion in the range of between the applied −side reference voltage REF− and +side reference voltage REF+. Specifically, the AD converter 54 executes the conversion so that the range of REF−-REF+ of the detection signal of the light detector 16 are X bits that are the conversion bit number of the AD converter 54.

If the light detector 16 completes detection of light quantities of all wavelengths (λ1-λm), the light detector 16 outputs an exposure end signal. If the input unit 38 receives the exposure end signal from the light detector 16 (step S308), the curvature calculator 50 acquires the curvature characteristic information of the sensor unit 12, which is stored in the memory 48, and the curvature calculator 50 calculates the curvature of each detection target 26 based on the acquired detection signal of the light detector 16 and this curvature characteristic information of the sensor unit 12 (step S309).

The process of creating the shape of the structure body in subsequent step S111 and the process of displaying the shape in step S112 are the same as in the first embodiment.

Thereafter, the operation from step S301 is repeated.

In this manner, a routine A of step S301 to step S112 is repeated. Thereby, the shape of the structure body corresponding to the displacement of the structure body can be updated and displayed on the display 58.

If the input unit 38 receives a curvature computation end signal from the input device 56 while the above routine A is being executed (step S320), the process of this flowchart is terminated.

### Fourth Embodiment

Next, a fourth embodiment of the present invention will be described. Here, different points from the above-described second embodiment will be described, and the same parts are denoted by like reference numerals and a description thereof is omitted.

In the shape calculating apparatus 10 according to the second embodiment, the method of changing the dynamic range of either the intensity of light that is input to the sensor unit 12 or the detection signal of the light detector 16 that is the electric signal generated by the light detector 16 based on the light output from the sensor unit 12 is implemented by using the variable quantity setting of any one of the light intensity of the light source 14, the exposure time of the light detector 16, and the sensitivity of the light detector 16.

By contrast, in a shape calculating apparatus 10 according to the fourth embodiment, the variable quantity settings of two or more of the light intensity of the light source 14, the exposure time of the light detector 16, and the sensitivity of the light detector 16 are combined and used. Thereby, the shape calculating apparatus 10 of the fourth embodiment can also change the dynamic ranges of both the intensity of light that is input to the sensor unit 12 and the detection signal of the light detector 16, which is the electric signal generated by the light detector 16 based on the light output from the sensor unit 12. In one method of combining the variable quantity settings, one arbitrary variable quantity setting is first implemented. When the implementation by this variable quantity setting becomes difficult, another variable quantity setting is implemented. Besides, it is also possible to combine two or more adjusting functions, so as to change the setting in turn among them.

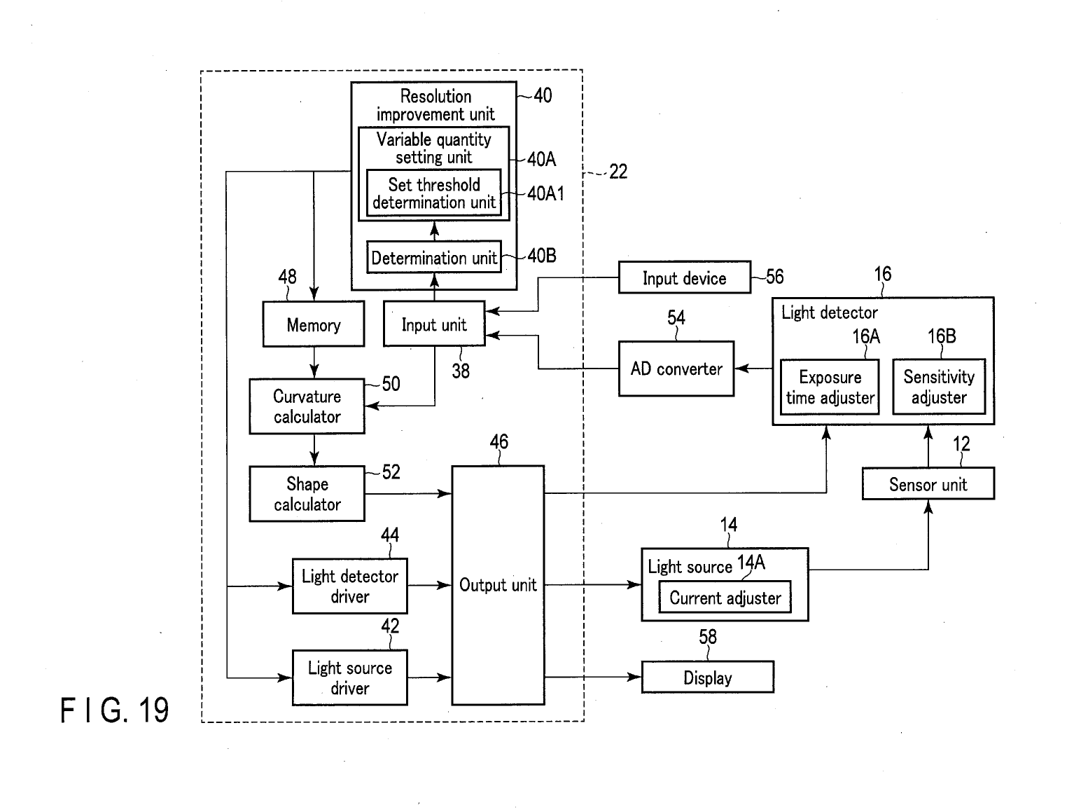

As shown in FIG. 19, in the shape calculating apparatus 10 according to the fourth embodiment, the variable quantity setting unit 40A of the resolution improvement unit 40 of the processor 22 includes a set threshold determination unit 40A1 configured to determine whether any one of the light intensity, exposure time, and sensitivity has exceeded an arbitrary threshold or not. The set threshold determination unit 40A1 first implements an arbitrary variable quantity setting, and then implements another variable quantity setting as needed. For example, the variable quantity setting by the current adjuster 14A of the light source 14 is first employed. While the setting is being executed by the variable quantity setting unit 40A, if the set threshold determination unit 40A1 determines a current set instruction value of the light source 14 has exceeded an arbitrary threshold, the variable quantity setting by the exposure time adjuster 16A of the light detector 16 is implemented. Alternatively, it may be configured that the variable quantity setting by the exposure time adjuster 16A or sensitivity adjuster 16B of the light detector 16 is first implemented, and another variable quantity setting is implemented as needed.

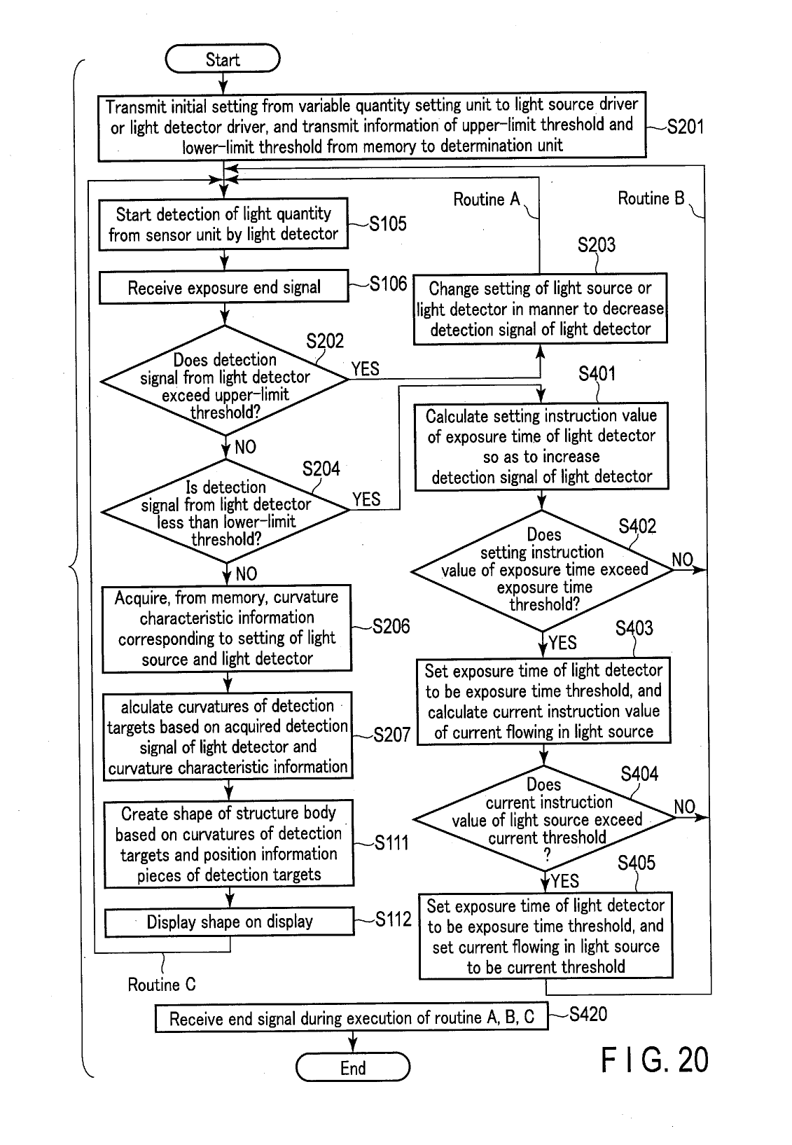

Hereinafter, the operation of the processor 22 of the shape calculating apparatus 10 according to the fourth embodiment will be described with reference to a flowchart of FIG. 20. In the example of this flowchart, the variable quantity setting by the exposure time adjuster 16A of the light detector 16 is first implemented, and the variable quantity setting by the current adjuster 14A is implemented as needed.

If the input unit 38 receives a curvature computation start signal from the input device 56, the operation of this flowchart is started. Here, the initial setting of step S201 and the routine A of step S105, step S106, step S202, and step S203 are the same as in the second embodiment.

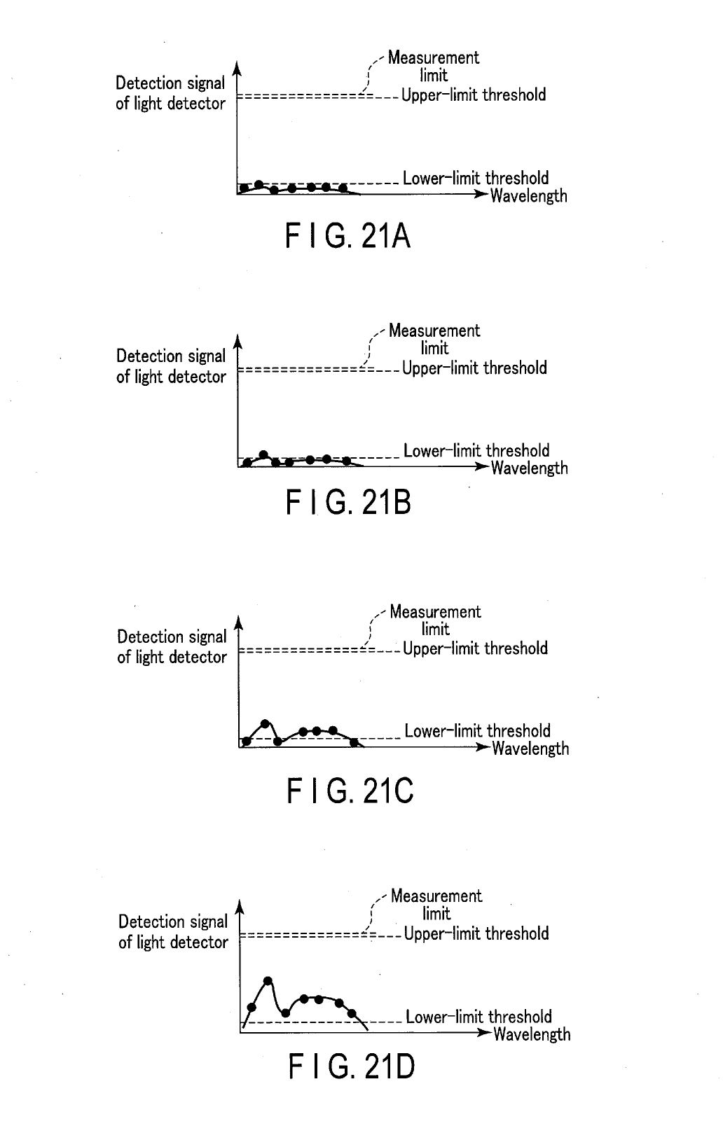

In step S204, if the determination unit 40B determines that the detection signal from the light detector 16 is less than the lower-limit threshold, as shown in FIG. 21A, the variable quantity setting unit 40A of the resolution improvement unit 40 in this embodiment calculates such a setting instruction value of the exposure time of the light detector 16 so as to increase the detection signal of the light detector 16 (step S401). For example, if the present setting instruction value of the exposure time is A, the variable quantity setting unit 40A calculates a setting instruction value B (B=A+ΔT) of exposure time in which a predetermined time ΔT is added to A. Then, the variable quantity setting unit 40A determines, by the set threshold determination unit 40A1, whether the calculated setting instruction value B of exposure time has exceeded an exposure time threshold ET or not (B<ET) (step S402). If the set threshold determination unit 40A1 determines that the calculated setting instruction value B of exposure time does not exceed the exposure time threshold ET, the variable quantity setting unit 40A transmits the calculated setting instruction value B of exposure time to the light detector driver 44, thereby changing the setting of the exposure time adjuster 16A of the light detector 16 to this exposure time. At this time, it may be configured that the setting of the light detector 16 is not only changed, but the lower-limit threshold that is the reference for determination of the determination unit 40B is also changed. Specifically, the lower-limit threshold can be changed to an optimal lower-limit threshold for the detection signal of the light detector 16 after the change of the setting. Thereafter, the operation returns to the process of step S105.

If the detection signal from the light detector 16 is still less than the lower-limit threshold, as shown in FIG. 21B, even after the exposure time has been increased, the operation advances from step S204 to the process of step 401 once again. Then, the variable quantity setting unit 40A of the resolution improvement unit 40 calculates once again such a setting instruction value of the exposure time of the light detector 16 so as to increase the detection signal of the light detector 16. At this time, the variable quantity setting unit 40A calculates a setting instruction value C (C=B+ΔT) of exposure time in which a predetermined time ΔT is added to the present setting instruction value B of exposure time. Then, in step 402, if it is determined that the setting instruction value C of exposure time does not exceed the exposure time threshold ET, the variable quantity setting unit 40A transmits the calculated setting instruction value C of exposure time to the light detector driver 44, thereby changing the setting of the exposure time adjuster 16A of the light detector 16 to this exposure time. At this time, it may be configured that the setting of the light detector 16 is not only changed, but the lower-limit threshold that is the reference for determination of the determination unit 40B is also changed. Thereafter, the operation returns to the process of step S105.

If the detection signal from the light detector 16 is still less than the lower-limit threshold, as shown in FIG. 21C, even after the exposure time has been increased once again, the operation advances from step S204 to the process of step 401 once again. Then, the variable quantity setting unit 40A of the resolution improvement unit 40 calculates once again such a setting instruction value of the exposure time of the light detector 16 so as to increase the detection signal of the light detector 16. At this time, the variable quantity setting unit 40A calculates a setting instruction value D (D=C+ΔT) of exposure time in which a predetermined time ΔT is added to the present setting instruction value C of exposure time. If the calculated setting instruction value D of exposure time exceeds the exposure time threshold ET, the set threshold determination unit 40A1 determines this in step S402. In this case, the variable quantity setting unit 40A sets the exposure time to be not D but the time of the exposure time threshold ET, and calculates a current instruction of a current flowing in the light source 14 (step S403). Specifically, since the setting change by the exposure time is not possible, the setting by the current is implemented. For example, if the present current instruction value of the light source 14 is Y, the variable quantity setting unit 40A calculates a current instruction value Z (Z=Y+ΔI) of the light source 14 in which a predetermined current ΔI is added to Y. Then, the variable quantity setting unit 40A determines, by the set threshold determination unit 40A1, whether the calculated current instruction value Z of the light source 14 has exceeded a current threshold IT or not (Z<IT) (step S404). If the set threshold determination unit 40A1 determines that the current instruction value Z of the light source 14 does not exceed the current threshold IT, the variable quantity setting unit 40A transmits the calculated current instruction value Z of the light source 14 to the light source driver 42, thereby changing the setting of the current adjuster 14A of the light source 14 to this current instruction value Z. At this time, it may be configured that the setting of the light source 14 is not only changed, but the lower-limit threshold that is the reference for determination of the determination unit 40B is also changed. Specifically, the lower-limit threshold can be changed to an optimal lower-limit threshold for the detection signal of the light detector 16 after the change of the setting. Thereafter, the operation returns to the process of step S105.

Besides, in step S404, if the set threshold determination unit 40A1 determines that the current instruction value Z of the light source 14 exceeds the current threshold IT, the variable quantity setting unit 40A sets the exposure time to be the time of the exposure time threshold ET, and also sets the value of the current flowing in the light source 14 to be the value of the current threshold IT (step S405). Specifically, if the setting change by the current is not possible either, the largest current is set, and no further setting change is executed. Thereafter, the operation returns to the process of step S105.

In this manner, a routine B of step S105, step S106, step S202, step S204, and step S401 to step S405 can be repeated. Specifically, when the detection signal of the light detector 16 is less than the lower-limit threshold even after the setting change, the setting of the exposure time adjuster 16A of the light detector 16 and the setting of the current adjuster 14A of the light source 14 are changed through the light detector driver 44 and light source driver 42 so that the detection signal of the light detector 16 further increases. In this manner, the setting change of the exposure time adjuster 16A of the light detector 16 and the setting change of the current adjuster 14A of the light source 14 can be executed stepwise so that the detection signal of the light detector 16 can be acquired with the optimal, setting.

Needless to say, when the setting change by the current not possible either, the variable quantity setting by the sensitivity adjuster 16B of the light detector 16 may be implemented.

In addition, if the determination unit 40B determines, in step S202, that the detection signal from the light detector 16 does not exceed the upper-limit threshold and if the determination unit 40B determines, in step S204, that the detection signal from the light detector 16 is not less than the lower-limit threshold, the operation advances from step S204 to the process of step S206. For example, the intensity of light emitted from the light source 14 is increased by increasing the driving current of the light source 14. Thereby, as shown in FIG. 21D, the detection signal from the light detector 16 exceeds the lower-limit value. The acquisition process of curvature characteristic information in step S206, the calculation process of the curvature of each detection target 26 in step S207, the process of creating the shape of the structure body in step S111, and the process of displaying the shape in step S112 are the same as in the second embodiment.

Thereafter, the operation from step S105 is repeated.

In this manner, a routine C of step S105 to step S112 is repeated. Thereby, the shape of the structure body corresponding to the displacement of the structure body can be updated and displayed on the display 58.

If the input unit 38 receives a curvature computation end signal from the input device 56 while the above routine A, routine B or routine C is being executed (step S420), the process of this flowchart is terminated.

As described above, the shape calculating apparatus 10 according to the fourth embodiment includes the setting change unit (resolution improvement function) configured to change the two dynamic ranges of both the intensity of light that is input to the sensor unit 12 and the detection signal of the light detector 16 that is the electric signal generated by the light detector 16 based on the light that is output from the sensor unit 12. Specifically, the setting change unit includes two or more of a set of the light source driver 42 and the current adjuster 14A of the light source 14; a set of the light detector driver 44 and the exposure time adjuster 16A of the light detector 16; and a set of the light detector driver 44 and the sensitivity adjuster 16B of the light detector 16. It is thus possible to execute the change by a combination of two or more of methods of changing the dynamic ranges.

Needless to say, it is also possible to combine the change relating to the digital conversion as described in the third embodiment.

Besides, the shape calculating apparatus 10 according to each of the first to fourth embodiments can be mounted in an endoscope. In the present specification, endoscopes are not limited to endoscopes for medical use and endoscopes for industrial use, and the endoscopes designate general equipment including an insertion section to be inserted into an insertion target.

Hereinafter, an endoscope for medical use will be described as an example of the endoscope.

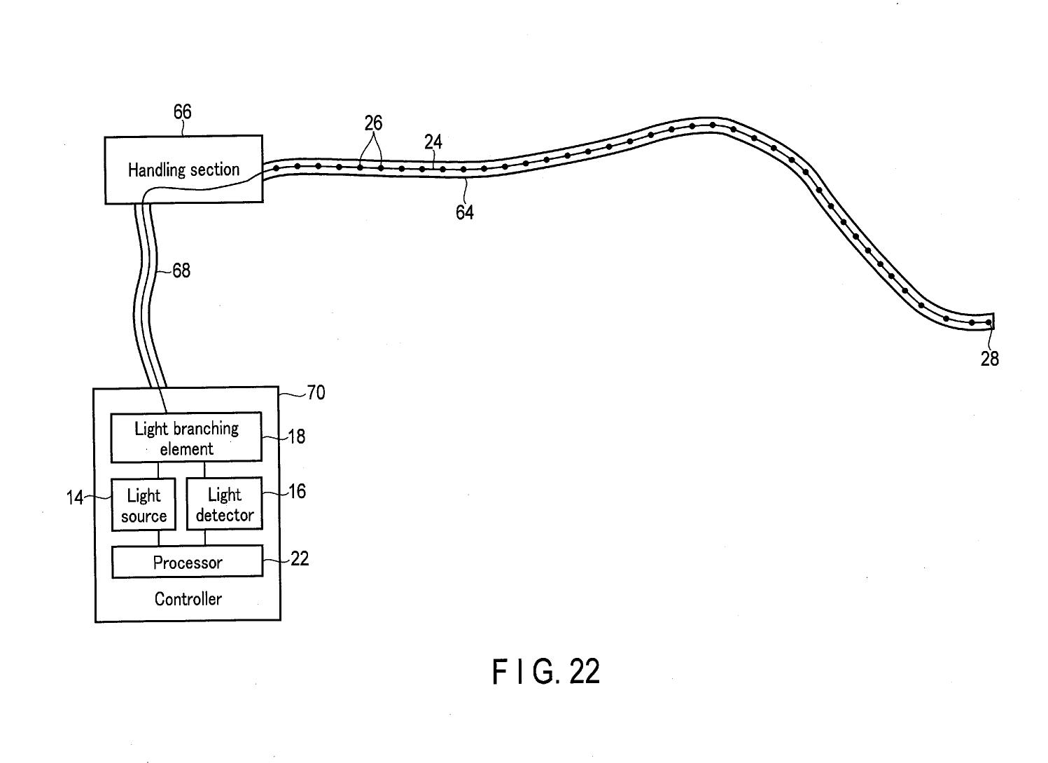

For example, FIG. 22 illustrates an endoscope system in which the light guide 24 of the shape calculating apparatus 10 according to the embodiment is disposed along an insertion section 64, or a structure body, of an endoscope. This endoscope system includes an endoscope, which is provided with an elongated insertion section 64 that is a structure body to be inserted into a subject (e.g. a body cavity (lumen cavity)), which is an observation target; a handling section 66 coupled to a proximal portion of the insertion section 64; and a connection cable 68. The endoscope system further includes a controller 70 configured to control the endoscope.

Here, the insertion section 64 includes, from the distal side toward the proximal side of the insertion section 64, a distal rigid section, an operation bendable section configured to bend, and a flexible tube section. The distal rigid section is a distal portion of the insertion portion 64, and is a rigid member. This distal rigid section is provided with an imager (not shown).

The operation bendable section bends in a desired direction in accordance with an operation by an endoscope operator (a worker such as a doctor) of a bend operation knob provided on the handling section 66. By operating the bend operation knob, the operator bends the operation bendable section. By the bending of the operation bendable section, the position and direction of the distal rigid section are varied, so that an observation target is captured in an observation view field that is an imaging range of the imager. Illumination light is radiated from an illumination window (not shown) provided in the distal rigid section on the captured observation target, and the observation target is illuminated. The operation bendable section is configured with node rings (not shown) being coupled along the longitudinal direction of the insertion section 64. The node rings swing relative to each other, and thereby the operation bendable section bends.

The flexible tube section has a desired flexibility, so as to be bent by external force. The flexible tube section is a tubular member extending from the handling section 66.

The connection cable 68 connects the handling section 66 and controller 70.

The controller 70 executes an image processing on an observation image captured by the imager of the endoscope, so as to cause a display (not shown) to display the observation image that has been subjected to the image processing. In the embodiment, as shown in FIG. 22, the light source 14, light detector 16, light branching element 18, and processor 22 of the shape calculating apparatus 10 are incorporated in the controller 70. The light guide 24 is disposed to extend in the longitudinal axial direction of the insertion section 64 from the controller 70 through the connection cable 68 and handling section 66. The reflection member 28 is provided in the distal rigid section of the insertion section 64. In this case, the detection targets 26 are provided at positions in portions of the light guide 24 that correspond to the operation bendable section and flexible tube section of the insertion section 64.

In the meantime, the structure body is not limited to this endoscope and may be various probes, catheters, over-sheaths (tubes used in assisting the insertion of endoscopes, catheters, etc.), and the like.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A shape calculating apparatus comprising:
a light source configured to emit light;
an optical fiber disposed in a structure body that is a shape calculation target, and configured to guide light emitted from the light source;
detection targets provided in the optical fiber along a longitudinal direction of the optical fiber, the detection targets having mutually different light absorption spectra, the detection targets being configured to absorb light propagated by the optical fiber in accordance with a bend shape of the optical fiber and to decrease a quantity of light;
a light detector configured to detect light quantity information at wavelengths included in the light absorption spectra of the detection targets, the light quantity information relating to light that is propagated by the optical fiber and the quantity of which is decreased by the detection targets;
a calculator configured to execute, based on the light quantity information, a calculation relating to a shape of each of the detection targets; and
a setting change unit configured to change, with respect to each of the wavelengths included in the light absorption spectra, a dynamic range of at least either an intensity of light that is input to the optical fiber or an electric signal generated by the light detector based on light that is output from the optical fiber.

2. The shape calculating apparatus of claim 1, wherein the setting change unit is configured to change the dynamic range of the electric signal by changing an exposure time of the light detector.

3. The shape calculating apparatus of claim 1, wherein the setting change unit is configured to change the dynamic range of the electric signal by changing a frequency of a synchronization signal relating to detection of the light detector.

4. The shape calculating apparatus of claim 1, wherein the setting change unit is configured to change the dynamic range of the electric signal by changing a detection sensitivity of the light detector.

5. The shape calculating apparatus of claim 1, wherein the setting change unit is configured to change the dynamic range of the electric signal by changing a range of a detection signal from the light detector.

6. The shape calculating apparatus of claim 5, wherein the change of the range of the detection signal from the light detector includes a change relating to digital conversion.

7. The shape calculating apparatus of claim 1, wherein the setting change unit is configured to change the dynamic range of the intensity of light that is input to the optical fiber by changing the intensity of the light that is input to the optical fiber.

8. The shape calculating apparatus of claim 2, wherein the setting change unit is configured to execute a change in which two or more methods of changing the dynamic range are combined.

9. The shape calculating apparatus of claim 2, further comprising a determination unit configured to determine whether the change of the dynamic range in the setting change unit is executed or not.

10. The shape calculating apparatus of claim 9, wherein the determination unit is configured to compare a detection signal of the light detector with a detection lower limit and a detection upper limit, determining that the change of the dynamic range is to be executed when the determination unit has determined that the detection signal is not in a detectable range.

11. The shape calculating apparatus of claim 10, further comprising a memory that stores thresholds relating to the detection lower limit and the detection upper limit.

12. The shape calculating apparatus of claim 9, further comprising an instruction unit configured to instruct a method of changing information relating to determination by the determination unit or changing the dynamic range by the setting change unit.

13. The shape calculating apparatus of claim 1, wherein the setting change unit is configured to execute the change of the dynamic range stepwise.

14. An endoscope system comprising:
an endoscope including an insertion section to be inserted in a subject;
a controller connected to the endoscope; and
the shape calculating apparatus of claim 1,
the optical fiber being provided in the insertion section of the endoscope,
the calculator being provided in the controller and configured to calculate a shape of the insertion section of the endoscope based on the light quantity information.
